(19) United States
(12) Patent Application Publication
(10) Pub. No.: US 2008/0089923 A1
(43) Pub. Date: Apr. 17, 2008

(54) Biodegradable ocular implants and methods for treating ocular conditions

(76) Inventors: Michael J. Burkstrand, Richfield, MN (US); Signe R. Erickson, Long Beach, CA (US); Stephen J. Chudzik, St. Paul, MN (US); Pamela J. Reed, St. Paul, MN (US)

Correspondence Address: KAGAN BINDER, PLLC, SUITE 200, MAPLE ISLAND BUILDING, 221 MAIN STREET NORTH, STILLWATER, MN, 55082, US

(21) Appl. No.: 11/904,805

(22) Filed: Sep. 28, 2007

Related U.S. Application Data
(60) Provisional application No. 60/848,563, filed on Sep. 29, 2006.

(51) Int. Cl. A61F 2/02; A61K 38/02; A61K 39/395; A61P 27/02

(52) U.S. Cl. 424/428; 424/130.1; 424/133.1; 514/2

(57) ABSTRACT

Biodegradable ocular implants are described. The ocular implants include a bioactive agent that can be released within the eye to treat an ocular condition or indication. The implants can be used for the administration of a bioactive agent over prolonged periods of time. In some aspects the implants are formed of a matrix of natural biodegradable polysaccharides.

### CROSS-REFERENCE TO RELATED APPLICATION

This application claims the benefit of U.S. Provisional Patent Application Ser. No. 60/848,563, filed Sep. 29, 2006, entitled OCULAR IMPLANTS INCLUDING NATURAL BIODEGRADABLE POLYSACCHARIDES AND METHODS FOR TREATING OCULAR CONDITIONS, the disclosure of which is incorporated herein by reference.

### TECHNICAL FIELD

The present invention relates to ocular implants comprising a biodegradable material and a bioactive agent. The bioactive agent can provide a therapeutic effect to treat on ocular condition.

### BACKGROUND

In recent years, much attention has been given to site-specific delivery of drugs within a patient. Although various drugs have been developed for treatment of a wide variety of ailments and diseases of the body, in many instances, such drugs cannot be effectively administered systemically without risk of detrimental side effects. Site-specific drug delivery focuses on delivering the drugs locally, i.e., to the area of the body requiring treatment. One benefit of the local release of bioactive agents is the avoidance of toxic concentrations of drugs that are at times necessary, when given systemically, to achieve therapeutic concentrations at the site where they are required.

Site-specific drug delivery can be accomplished by injection and/or implantation of an article or device that releases the drug to the treatment site. Injection of drugs can have limitations, for example, by requiring multiple administrations, increasing risk of complications (such as infection), and patient discomfort. Implantation of an article or device that delivers drug to the treatment site has therefore gained much interest in recent years.

Further, site-specific drug delivery has been enhanced by technologies that allow controlled release of one or more drugs from an implanted device or article. Controlled release can relate to the duration of time drug is released from the device or article, and/or the rate at which the drug is released.

Several challenges confront the use of medical devices or articles that release bioactive agents into a patient's body. For example, treatment may require release of the bioactive agent(s) over an extended period of time (for example, weeks, months, or even years), and it can be difficult to sustain the desired release rate of the bioactive agent(s) over such long periods of time. Further, the device or article surface is preferably biocompatible and non-inflammatory, as well as durable, to allow for extended residence within the body.

Generally speaking, a bioactive agent can be associated with the surface of a medical device or article by surface modification, embedded, and released from within polymeric materials (matrix-type), or surrounded by and released through a carrier (reservoir-type). The polymeric materials in such applications should optimally act as a biologically inert barrier and not induce further undesired tissues responses within the body, such as a strong inflammatory response. However, many polymers used in association with medical devices do not provide ideal properties when placed in the body.

Synthetic biodegradable polymers, such as polyglycolide-type molecules, have been used for the construction of implantable medical devices and for delivery of bioactive agents. While there has been an abundance of prior art relating to these devices, some concerns exist that regard the use of synthetic materials which degrade into materials that are not typically found in the body, or that are found at particularly low levels in the body. These types of biodegradable materials have the potential to degrade into products that cause unwanted side effects in the body by virtue of their presence or concentration in vivo. These unwanted side effects can include immune reactions, toxic buildup of the degradation products in the body, or the initiation or provocation of other adverse effects on cells or tissue in the body.

Another problem is that preparations of some biodegradable materials may not be obtained at consistent purity due to variations inherent in natural materials. This is relevant at least with regard to biodegradable materials derived from animal sources. Inconsistencies in preparations of biodegradable materials can result in problematic implantable devices.

Additional concerns are that preparations from animal sources may provide other unwanted contaminants, such as antigenic factors. These antigenic factors may promote a localized immune response in the vicinity of the implanted article and foul its function. These factors may also cause infection as well as local inflammation.

In addition, the delivery of bioactive agents within limited access regions of the body can present additional challenges. Limited access regions of the body can be characterized in terms of physical accessibility as well as therapeutic accessibility. For example, the relatively small size and sensitive tissues surrounding the eye can contribute to physical accessibility difficulties. In addition, ocular absorption of systemically administered pharmacologic agents is limited by the blood ocular barrier, namely the tight junctions of the retinal pigment epithelium and vascular endothelial cells. These can make accessing the eye with therapeutics difficult. High systemic doses of bioactive agents can penetrate this blood ocular barrier in relatively small amounts, but expose the patient to the risk of systemic toxicity. Intravitreal injection of bioactive agents (such as drugs) is an effective means of delivering a drug to the posterior segment of the eye in high concentrations. However, these repeated injections carry the risk of such complications as infection, hemorrhage, and retinal detachment. Patients also often find this procedure somewhat difficult to endure.

Because description of the invention will involve treatment of the eye as an illustrative embodiment, basic anatomy of the eye will now be described in some detail with reference to FIG. 1, which illustrates a cross-sectional view of the eye. Beginning from the exterior of the eye, the structure of the eye includes the iris 38 that surrounds the pupil 40. The iris 38 is a circular muscle that controls the size of the pupil 40 to control the amount of light allowed to enter the eye. A transparent external surface, the cornea 30, covers both the pupil 40 and the iris 38. Continuous with the cornea 30, and forming part of the supporting wall of the eyeball, is the sclera 28 (the white of the eye). The pars plana is a region of the eye approximately 4 mm posterior to the point on the globe where the colored iris 38 meets the white sclera 28. The pars plana encircles the iris and is not constant in width, but rather typically varies between 2-3 mm in width around the iris (with the largest width of the pars plana typically lying on the temporal side and measuring about 3 mm in width).

The conjunctiva 32 is a clear mucous membrane covering the sclera 28. Within the eye is the lens 20, which is a transparent body located behind the iris 38. The lens 20 is suspended by ligaments attached to the anterior portion of the ciliary body 21. Light rays are focused through the transparent cornea 30 and lens 20 upon the retina 24. The central point for image focus (the visual axis) in the human retina is the fovea (not shown in the figures). The optic nerve 42 is located opposite the lens.

There are three different layers of the eye, the external layer, formed by the sclera 28 and cornea 30; the intermediate layer, which is divided into two parts, namely the anterior (iris 38 and ciliary body 21) and posterior (the choroid 26); and the internal layer, or the sensory part of the eye, formed by the retina 24. The sclera 28 is composed of dense, fibrous tissue and is composed of collagen fiber. Scleral thickness is approximately 1 mm posteriorly near the optic nerve and approximately 0.3 mm anteriorly. At the pars plana, the eye tissues are composed of sclera only; there is no choroidal or retinal tissue layer within this region. For this reason, the avascular pars plana is typically selected for implantation and/or injection of materials into the interior (vitreous) of the eye.

The lens 20 divides the eye into the anterior segment (in front of the lens) and the posterior segment (behind the lens). More specifically, the eye is composed of two chambers of fluid: the anterior chamber 34 (between the cornea 30 and the iris 38), and the vitreous chamber 22 (between the lens 20 and the retina 24). The anterior chamber 34 is filled with aqueous humor whereas the vitreous chamber 22 is filled with a more viscous fluid, the vitreous humor.

The vitreous chamber 22 is the largest chamber of the eye, consisting of approximately 4.5 ml of fluid. The vitreous chamber is filled with a transparent gel composed of a random network of thin collagen fibers in a highly dilute solution of salts, proteins and hyaluronic acid (the vitreous humor comprises approximately 98% water).

### SUMMARY OF THE INVENTION

In one aspect, the present invention provides biodegradable implants that are particularly useful for delivering bioactive agents to a treatment site within a body. In particular, the biodegradable implants can be configured for placement and release of the bioactive agent in the interior of the eye. Upon implantation, bioactive agent can be released from the implant and provide a therapeutic effect at the treatment site. In particular, the biodegradable implants can be placed in a portion of the eye and are herein referred to as ocular implants.

According to experimental studies associated with the invention, small biodegradable ocular implants having a polypeptide agent were prepared and placed in the inner eye of a mammal in a minimally invasive manner. Pharmacokinetic analysis revealed that these implants were capable of releasing polypeptide to the vitreal fluid in amounts suitable for the treatment of ocular conditions. Notably, analysis also revealed that the implants released the polypeptide over a prolonged period of time after placement of implant in the eye (i.e., for periods of time of one about month or greater following implantation).

Explant analysis from the experimental studies also revealed that bioactive agent activity was maintained in the implant over the period of treatment. In view of this result, the implant not only provides a suitable matrix for the retention and release of a bioactive agent over these longer time periods, but also prevents loss of bioactive agent activity over the course of treatment.

Experimental studies also showed that implant formulations could be altered to adjust the delivery rate and the delivery period of the polypeptide from the implant, without compromising the bioactivity of the polypeptide. This “tunability” of the implant system provides great advantages for the treatment of ocular conditions requiring administration of bioactive agent over prolonged periods of time, and accommodates the preparation of implants having a wide variety of bioactive agents and bioactive agent release profiles.

In one aspect, the invention provides a biodegradable implant for delivery of a bioactive agent to the interior of the eye, wherein the implant comprises a matrix comprising a biodegradable polymer and a bioactive agent and is capable of releasing a therapeutically effective amount of bioactive agent in the interior of the eye after a period of about 30 days from implantation. In another aspect, the ocular implant is configured for delivery of a bioactive agent to the eye, wherein at least a portion of the bioactive agent is released from the implant after a period of implantation of about three months or greater.

The ocular implant can have certain dimensions desirable for delivering and/or immobilizing the implant to and/or at a target location in the eye. In many cases, the ocular implant of the invention can be delivered to the eye in a minimally invasive manner. In some aspects, the implant is sized so that the method of insertion does not require additional procedures to be performed during or after the insertion process, such as suturing of the sclera. Therefore, the implant is configured so that it can be placed at a location in the inner eye using a sutureless procedure. In some aspects the ocular implant is configured for placement within a needle having a size of 25 gauge or smaller.

In some aspects, the implant has an elongate shape. The elongate shape can be that of a rod, cylinder, or filament. In one specific embodiment, the ocular implant comprises a length of about 5 mm or less. In another specific embodiment, the ocular implant comprises a diameter of about 0.35 mm or less. For example, the ocular implant can have a cylindrical or rod-like shape, and the diameter of the implant is about 0.35 mm or less. In one specific embodiment, the ocular implant comprises a diameter of about 0.35 mm or less, and a length of about 5 mm or less. These dimensions can provide advantages for the insertion of the implant into a portion of the eye.

In another specific embodiment, the ocular implant has a weight of about 6 mg or less. In another specific embodiment, the ocular implant has a weight of about 2.5 mg or less.

The ocular implants can have a defined structure and can be formed by any suitable process, including molding, extruding, shaping, cutting, casting, and the like.

In some aspects, the biodegradable implants include a matrix of natural biodegradable polysaccharides and a bioactive agent. In preparing these types of ocular implants, a plurality of natural biodegradable polysaccharides are crosslinked to each other via coupling groups that are pendent from the natural biodegradable polysaccharide (i.e., one or more coupling groups are chemically bonded to the polysaccharide). In some aspects, the coupling group on the natural biodegradable polysaccharide is a polymerizable group. In a free radical polymerization reaction the polymerizable group can crosslink natural biodegradable polysaccharides together in the composition, thereby forming a natural biodegradable polysaccharide matrix. A bioactive agent useful for treating an ocular condition of indication is included within the matrix. The matrix can be in the form of an implant having a size and configuration for placement in a portion of the eye.

Ocular implants formed of natural biodegradable polysaccharides can be enzymatically degraded within a portion of the eye. These types of ocular implants also offer the advantage of being generally non-enzymatically hydrolytically stable. This is particularly advantageous for bioactive agent delivery since the bioactive agent can be released from the implant under conditions of enzyme-mediated degradation. The kinetics of bioactive agent release from the ocular implant of the present invention can provide an advantage over the release of drugs retained within systems prepared from synthetic biodegradable materials, such as poly(lactides).

Natural biodegradable polysaccharides include polysaccharide and/or polysaccharide derivatives that are obtained from natural sources, such as plants or animals. Exemplary natural biodegradable polysaccharides include amylose, maltodextrin, amylopectin, starch, dextran, hyaluronic acid, heparin, chondroitin sulfate, dermatan sulfate, heparan sulfate, keratan sulfate, dextran sulfate, pentosan polysulfate, and chitosan. Preferred polysaccharides are low molecular weight polymers that have little or no branching, such as those that are derived from and/or found in starch preparations, for example, amylose, maltodextrin, and polyalditol.

Because of the particular utility of the amylose, maltodextrin, and polyalditol polymers, in some aspects natural biodegradable polysaccharides are used that have an average molecular weight of 500,000 Da or less, 250,000 Da or less, 100,000 Da or less, or 50,000 Da or less. In some aspects the natural biodegradable polysaccharides have an average molecular weight of 500 Da or greater. In some aspects the natural biodegradable polysaccharides have an average molecular weight in the range of about 1000 Da to about 10,000 Da. Natural biodegradable polysaccharides of particular molecular weights can be obtained commercially or can be prepared, for example, by acid hydrolysis and/or enzymatic degradation of a natural biodegradable polysaccharide preparation, such as starch. The decision of using natural biodegradable polysaccharides of a particular size range may depend on factors such as the desired physical characteristics of the ocular implant, the desired rate of degradation of the implant, and the type of bioactive agent present in the implant.

The natural biodegradable polysaccharides that are used in accordance with the methods and compositions of the invention are readily available at a low cost and/or can be prepared easily using established techniques. This allows for a cost effective method of fabricating ocular implants.

The use of natural biodegradable polysaccharides, such as maltodextrin or amylose, provides many advantages when used for the formation of an ocular implant. Degradation of a natural biodegradable polysaccharide-containing ocular implant can result in the release of, for example, naturally occurring mono- or disaccharides, such as glucose, which are common components of bodily fluids, such as the vitreous humor. Furthermore, the use of natural biodegradable polysaccharides that degrade into common components found in bodily fluids, such as glucose, can be viewed as more acceptable than the use of synthetic biodegradable polysaccharides that degrade into non-natural compounds, or compounds that are found at very low concentrations in the body.

In some aspects of the invention, this advantageous feature is reflected in the use of natural biodegradable polysaccharides which are non-animal derived, such as amylose and maltodextrin, and that degrade into products that present little or no immunogenic or toxic risk to the individual. The invention provides improved, cost-efficient, natural biodegradable polysaccharide compositions for articles that can be used in a variety of treatments for the eye.

Another advantage of the invention is that the natural biodegradable polysaccharide-based ocular implant are more resistant to hydrolytic degradation than other biodegradable polymers, such as poly(lactides). Degradation of the matrices prepared from natural biodegradable polysaccharides of the invention are primarily enzyme-mediated, with minimal or no hydrolysis of the natural biodegradable polysaccharide occurring when a natural biodegradable polysaccharide-containing composition is prepared under ambient conditions. This allows the natural biodegradable polysaccharide-based ocular implant to remain substantially stable (for example, resistant to degradation) prior to placing the implant into a portion of the eye. For example, a natural biodegradable polysaccharide ocular implant can be manipulated in a non-biological, aqueous-based-medium without risk that the implant will prematurely degrade due to non-enzyme-meditated hydrolysis. Systems that are based on biodegradable polymers such as poly(lactide) or poly(lactide-co-glycolide) are subject to hydrolysis even at relatively neutral pH ranges (e.g., pH 6.5 to 7.5) and therefore do not offer this advantage. The properties of the polymer systems of the present invention provide ocular implant with improved storage characteristics.

In some aspects, the invention provides a bioactive agent-releasing biodegradable ocular implant comprising (i) a matrix of natural biodegradable polysaccharides (ii) and a bioactive agent within the matrix. The implant is configured to reside in a portion of the eye and comprises an amount of bioactive agent useful for treating an ocular condition or indication. The implant is prepared having a matrix of natural biodegradable polysaccharides that includes bioactive agent, wherein the matrix is slowly degradable in the presence of ocular fluids and/or tissues.

The ocular implant can be prepared having any suitable bioactive agent for the treatment of an ocular condition or indication. Illustrative bioactive agents include antiproliferative agents, anti-inflammatory agents, anti-angiogenic agents, hormonal agents, antibiotics, neurotrophic factors, or combinations thereof.

In some aspects, the implant includes a larger hydrophilic bioactive agent, such as a polypeptide, nucleic acid, polysaccharide, or combinations thereof. Viral particles and cells can also be included in the ocular implant. The implant provides a distinct advantage for delivering these larger bioactive agents. Comparatively, use of non-degrading drug delivery matrices may not allow delivery of these larger bioactive agents if too large to diffuse out of the matrix. However, an ocular implant that includes a matrix of natural biodegradable polysaccharides allows release of the bioactive agent upon degradation of the matrix. In some aspects of the invention, the ocular implant comprises a bioactive agent having a molecular weight of about 10,000 Da or greater.

In some aspects the ocular implant comprises a bioactive agent that is a high molecular weight compound and that is an inhibitor of angiogenesis. For example, the inhibitor can be selected from angiostatin, thrombospondin, anti-VEGF antibody, and anti-VEGF fragment. In some aspects the ocular implant comprises a bioactive agent that is a high molecular weight compound and a hormonal agent. For example, the bioactive agent could be ciliary neurotrophic factor or pigment endothelium derived growth factor.

The ocular implant can also include lower molecular weight compounds. In some aspects these compounds are held within the matrix of the implant in particulate form. For example, the bioactive agent can be present in the form of microparticles that are immobilized in the matrix of natural biodegradable polysaccharide. In some aspects the bioactive agent is an antiproliferative agent, such as 13-cis retinoic acid, retinoic acid derivatives, 5-fluorouracil, taxol, sirolimus (rapamycin), analogues of rapamycin, tacrolimus, ABT-578, everolimus, paclitaxel, taxane, or vinorelbine. In some aspects the bioactive agent is an anti-inflammatory agent such as hydrocortisone, hydrocortisone acetate, dexamethasone 21-phosphate, fluocinolone, medrysone, methylprednisolone, prednisolone 21-phosphate, prednisolone acetate, fluoromethalone, betamethasone, triamcinolone, or triamcinolone acetonide. In some aspects the bioactive agent is an inhibitor of angiogenesis such as anecortave acetate or a receptor tyrosine kinase antagonist.

A bioactive agent can also be included in an ocular implant prepared using a natural biodegradable polysaccharide that is modified with a hydrophobic moiety. The hydrophobic moiety can be used to provide a biodegradable matrix having hydrophobic properties. The hydrophobic moieties can be pendent from the polysaccharide chain. Exemplary hydrophobic moieties include fatty acids and derivatives thereof, and C₂-C₁₈ alkyl chains.

In some aspects of the invention, the bioactive agent can be coupled to and cleavable from the polysaccharide. For example, a bioactive agent can be covalently attached to the polysaccharide via an ester bond. Upon implantation into a portion of the eye, the bond can be hydrolyzed resulting in the release of the bioactive agent which provides a therapeutic effect. Illustrative therapeutically useful bioactive agents include butyric acid, valproic acid, retinoic acid, and the like.

The invention also provides a method for delivery of a bioactive agent, or more than one bioactive agent, to a subject for the treatment of an ocular condition or indication.

In one aspect, the invention provides a method for administering a bioactive agent to the inner eye, the method comprising the steps of (a) providing a biodegradable implant comprising a matrix comprising a biodegradable polymer and a bioactive agent, wherein the implant is configured so that it can be placed at a location in the inner eye, (b) implanting the implant in the inner eye, and (c) maintaining the implant in the inner eye, wherein the implant releases a therapeutically effective amount of bioactive agent in the inner eye after a period of 30 days from the step of implanting.

In some aspects, the step of implanting comprises implanting the implant in the inner eye using a sutureless procedure.

In another aspect, the invention provides a method comprising the steps of providing an ocular implant comprising (a) a matrix of natural biodegradable polysaccharides and (b) a bioactive agent within the matrix to a portion of the eye. The method also comprises a step of maintaining the implant in the portion of the eye for a period of time sufficient for the treatment of an ocular condition of indication.

Within the eye the ocular implant is exposed to a carbohydrase that promotes the degradation of the matrix and release of the bioactive agent. For example, an ocular implant including amylose and/or maltodextrin polymers can be exposed to an α-amylase to promote degradation of the implant and release of the bioactive agent. During the step of maintaining the ocular implant generally is eroded on its surface and releases bioactive agent. Release of bioactive agent occurs until the implant is completely degraded.

Desirably, the ocular implant releases the bioactive agent over a prolonged period of time to treat the ocular condition or indication. For example, the ocular implant can be maintained in the eye for a period of about three months or greater to provide treatment to the eye. This means that a portion of the ocular implant remains in the eye and is able to release bioactive agent after a period of three months. The lifetime of the ocular implant may be greater than three months, in the range of three to eighteen months, in the range of three to twelve months, or in the range of three to six months.

The ocular condition or indication can be one or more selected from retinal detachment; vascular occlusions; retinitis pigmentosa; proliferative vitreoretinopathy; diabetic retinopathy; inflammations such as uveitis, choroiditis, and retinitis; degenerative disease (such as age-related macular degeneration, also referred to as AMD); vascular diseases; and various tumor-related conditions, including those associated with neoplasms.

In yet further embodiments, the biodegradable medical article can be used post-operatively, for example, as a treatment to reduce or avoid potential complications that can arise from ocular surgery. In one such embodiment, the medical article can be provided to a patient after cataract surgical procedures, to assist in managing (for example, reducing or avoiding) post-operative inflammation.

In some aspects, the step of providing comprises placing the implant in contact with retinal tissue. For example, the method can include providing the implant to a subretinal location. In another aspect, the step of providing comprises placing the implant in the vitreous.

In some aspects, the method of treatment of an ocular condition or indication comprises delivering the ocular implant to a target location in the eye via an implant delivery instrument. In some desired modes of practice, the ocular implant is releasably associated with a distal end of the implant delivery instrument. The step of providing can include the sub-steps of (i) providing a system comprising a delivery instrument and the ocular implant releasably associated with a portion of the instrument (ii) inserting the ocular implant and a portion of the instrument into the eye, and (iii) actuating the instrument to release the ocular implant at a target location in the eye.

In some aspects of the invention, the implant is delivered to a portion of the eye using an implant delivery instrument having a distal end with an outer diameter of about 0.5 mm or less. This can be particularly beneficial when it is desirable to minimize the size of any incision in the body, thereby reducing or avoiding the use of sutures or other closure devices.

In other aspects, the invention provide a kit for placing a biodegradable implant in the interior of the eye, the kit comprising a biodegradable implant comprises a matrix comprising a biodegradable polymer and a bioactive agent which is capable of releasing a therapeutically effective amount of bioactive agent in the interior of the eye after a period of 30 days from implantation, and an insertion instrument to provide the implant to a target site within the eye.

### BRIEF DESCRIPTION OF THE DRAWINGS

FIG. 1 is an illustration of a cross-sectional view of the eye.

FIG. 2 is a graph of cumulative BSA release from maltodextrin-acrylate filaments treated with amylase, over a period of time.

FIG. 3 is a graph of cumulative absorbance values of active and total IgG Fab fragment release from maltodextrin-acrylate filaments treated with amylase, over a period of time.

FIG. 4 is a graph of cumulative absorbance values of active and total IgG release from a maltodextrin-acrylate filament treated with amylase and percent degradation of the filament, over a period of time.

FIG. 5 is a graph of mass loss of biodegradable implants after periods of time in vitro and in vivo.

FIG. 6 is a graph of mass loss of biodegradable implants after periods of time in vitro and in vivo.

FIG. 7 is a graph of amounts of active F(Ab) fragment from explanted biodegradable implants after periods of time in vivo.

FIG. 8 is a graph of amounts of active F(Ab) fragment released from biodegradable implants in the vitreous after periods of time in vivo.

FIG. 9 is a graph of amounts of total and active F(Ab) fragment released from biodegradable implants after periods of time in vitro.

FIG. 10 is a graph of amounts of active F(Ab) fragment from explanted biodegradable implants after periods of time in vivo.

FIG. 11 is a graph of mass of biodegradable implants remaining after periods of time in vivo.

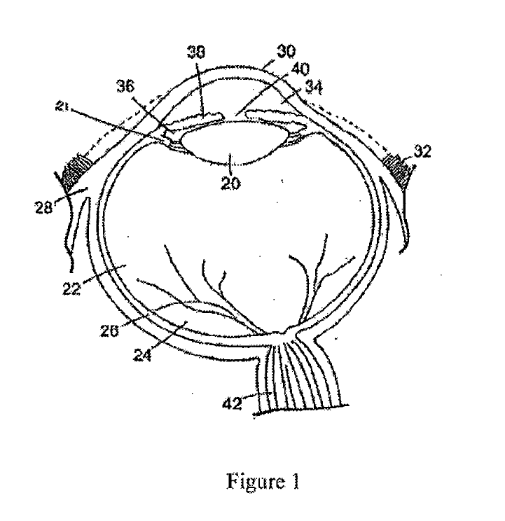

### DETAILED DESCRIPTION

The embodiments of the present invention described herein are not intended to be exhaustive or to limit the invention to the precise forms disclosed in the following detailed description. Rather, the embodiments are chosen and described so that others skilled in the art can appreciate and understand the principles and practices of the present invention.

All publications and patents mentioned herein are hereby incorporated by reference. The publications and patents disclosed herein are provided solely for their disclosure. Nothing herein is to be construed as an admission that the inventors are not entitled to antedate any publication and/or patent, including any publication and/or patent cited herein.

In some aspects, the polymeric compositions can be utilized in to form an ophthalmic article, such as an ocular implant. The ocular implant can be configured for placement at an internal site of the eye. Suitable ocular implants in accordance with these aspects can provide bioactive agent to any desired area of the eye. In some aspects, the ocular implant is utilized to deliver bioactive agent to a posterior segment of the eye (behind the lens). The biodegradable polysaccharide compositions described herein can be used for the formation of an ophthalmic article, such as an ocular implant.

In some aspects, the ocular implant can be configured for placement at a subretinal area within the eye. In some aspects the ocular implant is used in association with an ophthalmic devices. Ophthalmic devices are described in U.S. Patent Publication No. 2005/0143363 (“Method for Subretinal Administration of Therapeutics Including Steroids; Method for Localizing Pharmacodynamic Action at the Choroid and the Retina; and Related Methods for Treatment and/or Prevention of Retinal Diseases,” de Juan et al.); U.S. application Ser. No. 11/175,850 (“Methods and Devices for the Treatment of Ocular Conditions,” de Juan et al.); and related applications.

In some aspects, the invention provides a biodegradable implant that is formed from the biodegradable polysaccharide and that includes a bioactive agent, such as a high molecular weight bioactive agent useful for treating an ocular condition.

As referred to herein, a “natural biodegradable polysaccharide” refers to a non-synthetic polysaccharide that is capable of being enzymatically degraded but that is generally non-enzymatically hydrolytically stable. Natural biodegradable polysaccharides include polysaccharide and/or polysaccharide derivatives that are obtained from natural sources, such as plants or animals. Natural biodegradable polysaccharides include any polysaccharide that has been processed or modified from a natural biodegradable polysaccharide (for example, maltodextrin is a natural biodegradable polysaccharide that is processed from starch). Exemplary natural biodegradable polysaccharides include hyaluronic acid, starch, dextran, heparin, chondroitin sulfate, dermatan sulfate, heparan sulfate, keratan sulfate, dextran sulfate, pentosan polysulfate, and chitosan. Preferred polysaccharides are low molecular weight polymers that have little or no branching, such as those that are derived from and/or found in starch preparations, for example, amylose and maltodextrin. Therefore, the natural biodegradable polysaccharide can be a substantially non-branched or non-branched poly(glucopyranose) polymer.

Because of the particular utility of the amylose and maltodextrin polymers, it is preferred that natural biodegradable polysaccharides having an average molecular weight of 500,000 Da or less, 250,000 Da or less, 100,000 Da or less, or 50,000 Da or less. It is also preferred that the natural biodegradable polysaccharides have an average molecular weight of 500 Da or greater. A particularly preferred size range for the natural biodegradable polysaccharides is in the range of about 1000 Da to about 10,000 Da. Natural biodegradable polysaccharides of particular molecular weights can be obtained commercially or can be prepared. The decision of using natural biodegradable polysaccharides of a particular size range may depend on factors such as the physical characteristics of the composition (e.g., viscosity) used to form the implant, the desired rate of degradation of the implant, the presence of other components in the composition used to form the implant, for example, bioactive agents, etc.

As used herein, “amylose” or “amylose polymer” refers to a linear polymer having repeating glucopyranose units that are joined by α-1,4 linkages. Some amylose polymers can have a very small amount of branching via α-1,6 linkages (about less than 0.5% of the linkages) but still demonstrate the same physical properties as linear (unbranched) amylose polymers do. Generally amylose polymers derived from plant sources have molecular weights of about 1×10⁶ Da or less. Amylopectin, comparatively, is a branched polymer having repeating glucopyranose units that are joined by α-1,4 linkages to form linear portions and the linear portions are linked together via α-1,6 linkages. The branch point linkages are generally greater than 1% of the total linkages and typically 4%-5% of the total linkages. Generally amylopectin derived from plant sources have molecular weights of 1×10⁷ Da or greater.

Amylose can be obtained from, or is present in, a variety of sources. Typically, amylose is obtained from non-animal sources, such as plant sources. In some aspects, a purified preparation of amylose is used as starting material for the preparation of the amylose polymer having coupling groups. In other aspects, as starting material, amylose can be used in a mixture that includes other polysaccharides.

For example, in some aspects, starch preparations having a high amylose content, purified amylose, synthetically prepared amylose, or enriched amylose preparations can be used in the preparation of amylose having the coupling groups. In starch sources, amylose is typically present along with amylopectin, which is a branched polysaccharide. According to the invention, it is preferred to use compositions that include amylose, wherein the amylose is present in the composition in an amount greater than amylopectin, if present in the composition. For example, in some aspects, starch preparations having high amylose content, purified amylose, synthetically prepared amylose, or enriched amylose preparations can be used in the preparation of amylose polymer having the coupling groups. In some embodiments the composition includes a mixture of polysaccharides including amylose wherein the amylose content in the mixture of polysaccharides is 50% or greater, 60% or greater, 70% or greater, 80% or greater, or 85% or greater by weight. In other embodiments the composition includes a mixture of polysaccharides including amylose and amylopectin and wherein the amylopectin content in the mixture of polysaccharides is 30% or less, or 15% or less.

In some cases it may be desirable to use non-retrograding starches, such as waxy starch, in the current invention. The amount of amylopectin present in a starch may also be reduced by treating the starch with amylopectinase, which cleaves α-1,6 linkages resulting in the debranching of amylopectin into amylose.

In some cases a synthesis reaction can be carried out to prepare an amylose polymer having pendent coupling groups (for example, amylose with pendent ethylenically unsaturated groups) and steps may be performed before, during, and/or after the synthesis to enrich the amount of amylose, or purify the amylose.

Amylose of a particular size, or a combination of particular sizes can be used. In some embodiments amylose having an average molecular weight of 500,000 Da or less, 250,000 Da or less, 100,000 Da or less, 50,000 Da or less, preferably greater than 500 Da, or preferably in the range of about 1000 Da to about 10,000 Da is used. Amylose of particular molecular weights can be obtained commercially or can be prepared. For example, synthetic amyloses with average molecular masses of 70, 110,320, and 1,000 kDa can be obtained from Nakano Vinegar Co., Ltd. (Aichi, Japan). The decision of using amylose of a particular size range may depend on factors such as the physical characteristics of the composition (e.g., viscosity) used to form the implant, the desired rate of degradation of the implant, the presence of other component in the composition used to form the implant (for example, bioactive agents, etc.), etc.

Maltodextrin is typically generated by hydrolyzing a starch slurry with heat-stable α-amylase at temperatures at 85-90° C. until the desired degree of hydrolysis is reached and then inactivating the α-amylase by a second heat treatment. The maltodextrin can be purified by filtration and then spray dried to a final product. Maltodextrins are typically characterized by their dextrose equivalent (DE) value, which is related to the degree of hydrolysis defined as: DE=MW dextrose/number-averaged MW starch hydrolysate×100.

A starch preparation that has been totally hydrolyzed to dextrose (glucose) has a DE of 100, where as starch has a DE of about zero. A DE of greater than 0 but less than 100 characterizes the mean-average molecular weight of a starch hydrolysate, and maltodextrins are considered to have a DE of less than 20. Maltodextrins of various molecular weights, for example, in the range of about 500-5000 Da are commercially available (for example, from CarboMer, San Diego, Calif.).

In some aspects, the ocular implant can include a natural biodegradable non-reducing polysaccharide. The ocular implant can include a matrix having a plurality of natural biodegradable non-reducing polysaccharides along with a bioactive agent, such as a polypeptide. A non-reducing polysaccharide can provide an inert matrix thereby improving the stability of sensitive bioactive agents, such as proteins and enzymes. A non-reducing polysaccharide refers to a polymer of non-reducing disaccharides (two monosaccharides linked through their anomeric centers) such as trehalose (α-D-glucopyranosyl α-D-glucopyranoside) and sucrose (β-D-fructofuranosyl α-D-glucopyranoside). An exemplary non-reducing polysaccharide comprises polyalditol which is available from GPC (Muscatine, Iowa). In another aspect, the polysaccharide is a glucopyranosyl polymer, such as a polymer that includes repeating (1→3)O-β-D-glucopyranosyl units. Biodegradable non-reducing polysaccharides can be useful for formulating ocular implants that release the bioactive agent over a prolonged period of time, such as about three months or greater.

Refinement of the molecular weight of a polysaccharide preparation can be carried out using diafiltration. Diafiltration of polysaccharides such as maltodextrin can be carried out using ultrafiltration membranes with differing pore sizes. As an example, use of one or more cassettes with molecular weight cut-off membranes in the range of about 1 K to about 30 K can be used in a diafiltration process to provide polysaccharide preparations with average molecular weights in the range of less than 30 K Da, in the range of about 5 K Da to about 30 K Da, in the range of about 10 K Da to about 30 K Da, or in the range of about 1 K Da to about 10 K Da.

In some aspects, the ocular implant can include natural biodegradable polysaccharides that include chemical modifications other than the pendent coupling group. To exemplify this aspect, modified amylose having esterified hydroxyl groups can be prepared and used in compositions in association with the implants and methods of the invention. Other natural biodegradable polysaccharides having hydroxyl groups may be modified in the same manner. These types of modifications can change or improve the properties of the natural biodegradable polysaccharide making for an implant composition that is particularly suitable for a desired application. Many chemically modified amylose polymers, such as chemically modified starch, have at least been considered acceptable food additives.

As used herein, “modified natural biodegradable polysaccharides” refers to chemical modifications to the natural biodegradable polysaccharide that are different than those provided by the coupling group or the initiator group. Modified amylose polymers having a coupling group (and/or initiator group) can be used to form the ocular implants of the invention.

To exemplify this aspect, modified amylose is described. By chemically modifying the hydroxyl groups of the amylose, the physical properties of the amylose can be altered. The hydroxyl groups of amylose allow for extensive hydrogen bonding between amylose polymers in solution and can result in viscous solutions that are observed upon heating and then cooling amylose-containing compositions such as starch in solution (retrograding). The hydroxyl groups of amylose can be modified to reduce or eliminate hydrogen bonding between molecules thereby changing the physical properties of amylose in solution.

Therefore, in some embodiments the natural biodegradable polysaccharides, such as amylose, can include one or more modifications to the hydroxyl groups wherein the modifications are different than those provided by coupling group. Modifications include esterification with acetic anhydride (and adipic acid), succinic anhydride, 1-octenylsuccinic anhydride, phosphoryl chloride, sodium trimetaphosphate, sodium tripolyphosphate, and sodium monophosphate; etherification with propylene oxide, acid modification with hydrochloric acid and sulfinuric acids; and bleaching or oxidation with hydrogen peroxide, peracetic acid, potassium permanganate, and sodium hypochlorite.

Examples of modified amylose polymers include carboxymethyl amylose, carboxyethyl amylose, ethyl amylose, methyl amylose, hydroxyethyl amylose, hydroxypropyl amylose, acetyl amylose, amino alkyl amylose, allyl amylose, and oxidized amylose. Other modified amylose polymers include succinate amylose and oxtenyl succinate amylose.

In another aspect of the invention, the natural biodegradable polysaccharide is modified with a hydrophobic moiety in order to provide a biodegradable matrix having hydrophobic properties. Exemplary hydrophobic moieties include those previously listed, fatty acids and derivatives thereof, and C₂-C₁₈ alkyl chains. A polysaccharide, such as amylose or maltodextrin, can be modified with a compound having a hydrophobic moiety, such as a fatty acid anhydride. The hydroxyl group of a polysaccharide can also cause the ring opening of lactones to provide pendent open-chain hydroxy esters.

In some aspects, the hydrophobic moiety pendent from the natural biodegradable has properties of a bioactive agent. The hydrophobic moiety can be hydrolyzed from the natural biodegradable polymer and released from the matrix to provide a therapeutic effect. One example of a therapeutically useful hydrophobic moiety is butyric acid, which has been shown to elicit tumor cell differentiation and apoptosis, and is thought to be useful for the treatment of cancer and other blood diseases. Other illustrative hydrophobic moieties include valproic acid and retinoic acid. Retinoic acid is known to possess antiproliferative effects and is thought to be useful for treatment of proliferative vitreoretinopathy (PVR). The hydrophobic moiety that provides a therapeutic effect can also be a natural compound (such as butyric acid, valproic acid, and retinoic acid). Therefore, degradation of the matrix having a coupled therapeutic agent can produce natural degradation products.

In further aspects, the natural biodegradable polysaccharide can be modified with a corticosteroid. In these aspects, a corticosteroid, such as triamcinolone, can be coupled to the natural biodegradable polymer. One method of coupling triamcinolone to a natural biodegradable polymer is by employing a modification of the method described in Cayanis, E. et al., Generation of an Auto-anti-idiotypic Antibody that Binds to Glucocorticoid Receptor, The Journal of Biol. Chem., 261(11): 5094-5103 (1986). Triamcinolone hexanoic acid is prepared by reaction of triamcinolone with ketohexanoic acid; an acid chloride of the resulting triamcinolone hexanoic acid can be formed and then reacted with the natural biodegradable polymer, such as maltodextrin or polyalditol, resulting in pendent triamcinolone groups coupled via ester bonds to the natural biodegradable polymer.

Optionally, when the natural biodegradable polymer includes a pendent hydrophobic moiety and/or corticosteroid, an enzyme, such as lipase, can be used in association with the implant to accelerate degradation of the bond between the hydrophobic moiety and the polysaccharide (e.g., ester bond).

According to the invention, a natural biodegradable polysaccharide that includes a coupling group is used to form the ocular implant. Other polysaccharides can also be present in the composition. For example, the two or more natural biodegradable polysaccharides are used to form the ocular implant. Examples include amylose and one or more other natural biodegradable polysaccharide(s), and maltodextrin and one or more other natural biodegradable polysaccharide(s); in one aspect the composition includes a mixture of amylose and maltodextrin, optionally with another natural biodegradable polysaccharide.

In one preferred embodiment, amylose or maltodextrin is the primary polysaccharide. In some embodiments, the composition includes a mixture of polysaccharides including amylose or maltodextrin and the amylose or maltodextrin content in the mixture of polysaccharides is 50% or greater, 60% or greater, 70% or greater, 80% or greater, or 85% or greater by weight.

Purified or enriched amylose preparations can be obtained commercially or can be prepared using standard biochemical techniques such as chromatography. In some aspects, high-amylose cornstarch can be used.

As used herein, “coupling group” can include (1) a chemical group that is able to form a reactive species that can react with the same or similar chemical group to form a bond that is able to couple the natural biodegradable polysaccharides together (for example, wherein the formation of a reactive species can be promoted by an initiator); or (2) a pair of two different chemical groups that are able to specifically react to form a bond that is able to couple the natural biodegradable polysaccharides together. The coupling group can be attached to any suitable natural biodegradable polysaccharide, including the amylose and maltodextrin polymers as exemplified herein.

Contemplated reactive pairs include Reactive Group A and corresponding Reactive Group B as shown in the Table 1 below. For the preparation of an implant composition, a reactive group from group A can be selected and coupled to a first set of natural biodegradable polysaccharides and a corresponding reactive group B can be selected and coupled to a second set of natural biodegradable polysaccharides. Reactive groups A and B can represent first and second coupling groups, respectively. At least one and preferably two, or more than two reactive groups are coupled to an individual natural biodegradable polysaccharide polymer. The first and second sets of natural biodegradable polysaccharides can be combined and reacted, for example, thermochemically, if necessary, to promote the coupling of natural biodegradable polysaccharides and the formation of a natural biodegradable polysaccharide matrix.

TABLE 1

| Reactive group A | Reactive group B |
|---|---|
| amine hydroxyl, sulfhydryl | N-oxysuccinimide (“NOS”) |
| amine | Aldehyde |
| amine | Isothiocyanate |
| amine, sulfhydryl | Bromoacetyl |
| amine, sulfhydryl | Chloroacetyl |
| amine, sulfhydryl | Iodoacetyl |
| amine, hydroxyl | Anhydride |
| aldehyde | Hydrazide |
| amine, hydroxyl, carboxylic acid | Isocyanate |
| amine, sulfhydryl | Maleimide |
| sulfhydryl | Vinylsulfone |

Amine also includes hydrazide (R—NH—NH₂)

For example, a suitable coupling pair would be a natural biodegradable polysaccharide having an electrophilic group and a natural biodegradable polysaccharide having a nucleophilic group. An example of a suitable electrophilic-nucleophilic pair is N-hydroxysuccinimide-amine pair, respectively. Another suitable pair would be an oxirane-amine pair.

In some aspects, the natural biodegradable polysaccharides of the invention include at least one, and more typically more than one, coupling group per natural biodegradable polysaccharide, allowing for a plurality of natural biodegradable polysaccharides to be coupled in linear and/or branched manner. In some preferred embodiments, the natural biodegradable polysaccharide includes two or more pendent coupling groups.

In some aspects, the coupling group on the natural biodegradable polysaccharide is a polymerizable group. In a free radical polymerization reaction the polymerizable group can couple natural biodegradable polysaccharides together in the composition, thereby forming a biodegradable natural biodegradable polysaccharide matrix.

A preferred polymerizable group is an ethylenically unsaturated group. Suitable ethylenically unsaturated groups include vinyl groups, acrylate groups, methacrylate groups, ethacrylate groups, 2-phenyl acrylate groups, acrylamide groups, methacrylamide groups, itaconate groups, and styrene groups. Combinations of different ethylenically unsaturated groups can be present on a natural biodegradable polysaccharide, such as amylose or maltodextrin.

In preparing the natural biodegradable polysaccharide having pendent coupling groups any suitable synthesis procedure can be used. Suitable synthetic schemes typically involve reaction of, for example, hydroxyl groups on the natural biodegradable polysaccharide, such as amylose or maltodextrin. Synthetic procedures can be modified to produce a desired number of coupling groups pendent from the natural biodegradable polysaccharide backbone. For example, the hydroxyl groups can be reacted with a coupling group-containing compound or can be modified to be reactive with a coupling group-containing compound. The number and/or density of acrylate groups can be controlled using the present method, for example, by controlling the relative concentration of reactive moiety to saccharide group content.

In some modes of practice, the biodegradable polysaccharides have an amount of pendent coupling groups of about 0.7 μmoles of coupling group per milligram of natural biodegradable polysaccharide. In a preferred aspect, the amount of coupling group per natural biodegradable polysaccharide is in the range of about 0.3 μmoles/mg, or about 0.4 μmoles/mg, to about 0.7 μmoles/mg. For example, amylose or maltodextrin can be reacted with an acrylate groups-containing compound to provide an amylose or maltodextrin macromer having a acrylate group load level in the range of about 0.3 μmoles/mg, or about 0.4 μmoles/mg, to about 0.7 μmoles/mg.

As used herein, an “initiator” refers to a compound, or more than one compound, that is capable of promoting the formation of a reactive species from the coupling group. For example, the initiator can promote a free radical reaction of natural biodegradable polysaccharide having a coupling group. In one embodiment the initiator is a photoreactive group (photoinitiator) that is activated by radiation. In some embodiments, the initiator can be an “initiator polymer” that includes a polymer having a backbone and one or more initiator groups pendent from the backbone of the polymer.

In some aspects the initiator is a compound that is light sensitive and that can be activated to promote the coupling of the polysaccharides via a free radical polymerization reaction. These types of initiators are referred to herein as “photoinitiators.” In some aspects it is preferred to use photoinitiators that are activated by light wavelengths that have no or a minimal effect on a bioactive agent if present in the composition. A photoinitiator can be present in a composition independent of the polysaccharides or pendent from the polysaccharides.

In some embodiments, photoinitiation occurs using groups that promote an intra- or intermolecular hydrogen abstraction reaction. This initiation system can be used without additional energy transfer acceptor molecules and utilizing nonspecific hydrogen abstraction, but is more commonly used with an energy transfer acceptor, typically a tertiary amine, which results in the formation of both aminoalkyl radicals and ketyl radicals. Examples of molecules exhibiting hydrogen abstraction reactivity and useful in a polymeric initiating system, include analogs of benzophenone, thioxanthone, and camphorquinone.

In some preferred embodiments the photoinitiator includes one or more charged groups. The presence of charged groups can increase the solubility of the photoinitiator (which can contain photoreactive groups such as aryl ketones) in an aqueous system and therefore provide for an improved composition. Suitable charged groups include, for example, salts of organic acids, such as sulfonate, phosphonate, carboxylate, and the like, and onium groups, such as quaternary ammonium, sulfonium, phosphonium, protonated amine, and the like. According to this embodiment, a suitable photoinitiator can include, for example, one or more aryl ketone photogroups selected from acetophenone, benzophenone, anthraquinone, anthrone, anthrone-like heterocycles, and derivatives thereof; and one or more charged groups, for example, as described herein. Examples of these types of water-soluble photoinitiators have been described in U.S. Pat. No. 6,077,698.

In some aspects the photoinitiator is a compound that is activated by long-wavelength ultraviolet (UV) and visible light wavelengths. For example, the initiator includes a photoreducible or photo-oxidizable dye. Photoreducible dyes can also be used in conjunction with a compound such as a tertiary amine. The tertiary amine intercepts the induced triplet producing the radical anion of the dye and the radical cation of the tertiary amine. Examples of molecules exhibiting photosensitization reactivity and useful as an initiator include acridine orange, camphorquinone, ethyl eosin, eosin Y, erythrosine, fluorescein, methylene green, methylene blue, phloxime, riboflavin, rose bengal, thionine, and xanthine dyes. Use of these types of photoinitiators can be particularly advantageous when a light-sensitive bioactive agent is included in the implant.

Thermally reactive initiators can also be used to promote the polymerization of natural biodegradable polymers having pendent coupling groups. Examples of thermally reactive initiators include 4,4′ azobis(4-cyanopentanoic acid), 2,2-azobis[2-(2-imidazolin-2-yl)propane]dihydrochloride, and analogs of benzoyl peroxide. Redox initiators can also be used to promote the polymerization of the natural biodegradable polymers having pendent coupling groups. In general, combinations of organic and inorganic oxidizers, and organic and inorganic reducing agents are used to generate radicals for polymerization. A description of redox initiation can be found in Principles of Polymerization. 2^{nd} Edition, Odian G., John Wiley and Sons, pgs 201-204, (1981).

The ocular implant can also be formed using an initiator that includes an oxidizing agent/reducing agent pair, a “redox pair,” to drive polymerization of the biodegradable polysaccharide. In this case, polymerization of the biodegradable polysaccharide is carried out upon combining one or more oxidizing agents with one or more reducing agents. Other compounds can be included in the composition to promote polymerization of the biodegradable polysaccharides.

In order to promote polymerization of the biodegradable polysaccharides in a composition to form an ocular implant, the oxidizing agent is added to the reducing agent in the presence of the one or more biodegradable polysaccharides. For example, a composition including a biodegradable polysaccharide and a reducing agent is added to a composition including an oxidizing agent, or a composition including a biodegradable polysaccharide and an oxidizing agent is added to a composition containing a reducing agent. One desirable method of preparing an ocular implant is to combine a composition including a biodegradable polysaccharide and an oxidizing agent with a composition including a biodegradable polysaccharide and a reducing agent. For purposes of describing this method, the terms “first composition” and “second composition” can be used.

The oxidizing agent can be selected from inorganic or organic oxidizing agents, including enzymes; the reducing agent can be selected from inorganic or organic reducing agents, including enzymes. Exemplary oxidizing agents include peroxides, including hydrogen peroxide, metal oxides, and oxidases, including glucose oxidase. Exemplary reducing agents include salts and derivatives of electropositive elemental metals such as Li, Na, Mg, Fe, Zn, Al, and reductases. In one mode of practice, the reducing agent is present at a concentration of about 2.5 mM or greater when the reducing agent is mixed with the oxidizing agent. Prior to mixing, the reducing agent can be present in a composition at a concentration of, for example, 5 mM or greater.

Other reagents can be present in the composition to promote polymerization of the biodegradable polysaccharide. Other polymerization promoting compounds can be included in the composition, such as metal or ammonium salts of persulfate.

Optionally, the compositions and methods of the invention can include polymerization accelerants that can improve the efficiency of polymerization. Examples of useful accelerants include N-vinyl compounds, particularly N-vinyl pyrrolidone and N-vinyl caprolactam. Such accelerants can be used, for instance, at a concentration of between about 0.01% and about 5%, and preferably between about 0.05% and about 0.5%, by weight, based on the volume of the composition.

In some aspects, a natural biodegradable polysaccharide that includes a coupling group is used to form an ocular implant. Other polysaccharides can also be present in the ocular implant. For example, the ocular implant can include two different natural biodegradable polysaccharides, or more than two different natural biodegradable polysaccharides. For example, in some cases the natural biodegradable polysaccharide (such as amylose or maltodextrin) can be present in the ocular implant along with another biodegradable polymer (i.e., a secondary polymer), or more than one other biodegradable polymer. An additional polymer or polymers can be used to alter the properties of the matrix, or serve as bulk polymers to alter the volume of the matrix. For example, other biodegradable polysaccharides can be used in combination with the amylose polymer. These include hyaluronic acid, dextran, starch, amylose (for example, non-derivitized), amylopectin, cellulose, xanthan, pullulan, chitosan, pectin, inulin, alginates, and heparin.

The invention also provides methods of preparing ocular implants. The ocular implants can function as bioactive agent-releasing implants or depots. In some aspects, the ocular implants of the invention biodegrade within a period that is acceptable for the desired application.

The concentration of the natural biodegradable polysaccharide in the composition can be chosen to provide an ocular implant having a desired density of crosslinked natural biodegradable polysaccharide. In some embodiments, the concentration of natural biodegradable polysaccharide in the composition can depend on the type or nature of the bioactive agent that is included in the composition.

For example, in forming a implant, the concentration of the natural biodegradable polysaccharide may be higher to provide a more structurally rigid implant. Also, wherein it is desired to prepare an ocular implant having a prolonged rate of degradation, a composition having a relatively high concentration of polysaccharide is prepared.

In some embodiments, the natural biodegradable polysaccharide having the coupling groups is present in a composition used to form the ocular implant at a concentration of at least about 4.8% solids (50 mg polysaccharide+1 mL solution).

In more specific aspects the ocular implant is prepared using a composition having a concentration of polysaccharide of about 50% solids or greater, about 52.4% solids or greater, about 54.5% or greater, about 56.5% solids or greater, about 58.3% solids or greater, or about 60% solids.

In some aspects the ocular implant comprises a matrix prepared from a natural biodegradable polysaccharide comprising a molecular weight of about 50 KDa or less. In some aspects the ocular implant comprises a matrix prepared from a natural biodegradable polysaccharide having coupling groups pendent from the polysaccharide in an amount of about 0.4 mmol/mg polysaccharide or greater. In some aspects the implant is prepared using a composition having a concentration of polysaccharide of about 48.7% solids (950 mg polysaccharide+1 mL solution) or greater.

Other polymers or non-polymeric compounds can be included in the composition that can change or improve the properties of the ocular implant. These optional compounds can change the elasticity, flexibility, wettability, or adherent properties, (or combinations thereof) of the ocular implant.

Exemplary optional components include a mixture one or a combination of plasticizing agents. Suitable plasticizing agents include glycerol, diethylene glycol, sorbitol, sorbitol esters, maltitol, sucrose, fructose, invert sugars, corn syrup, and mixtures thereof. The amount and type of plasticizing agents can be readily determined using known standards and techniques.

The ocular implant of the present invention can also have can also be prepared by assembling an article having two or more “parts” wherein at least one of the parts has a matrix of biodegradable material. All or a portion of the ocular implant can be biodegradable. Desirably, for many applications, the ocular implant is entirely degradable.

The term “bioactive agent” refers to a peptide, protein, carbohydrate, nucleic acid, lipid, polysaccharide, synthetic inorganic or organic molecule, viral particle, cell, or combinations thereof, that causes a biological effect when administered in vivo to an animal, including but not limited to birds and mammals, including humans. Nonlimiting examples are antigens, enzymes, hormones, receptors, peptides, and gene therapy agents. Examples of suitable gene therapy agents include (a) therapeutic nucleic acids, including antisense DNA, antisense RNA, and interference RNA, and (b) nucleic acids encoding therapeutic gene products, including plasmid DNA and viral fragments, along with associated promoters and excipients.

Although not limited to such, the ocular implants of the invention are particularly useful for delivering bioactive agents that are large hydrophilic molecules, such as polypeptides (including proteins and peptides), nucleic acids (including DNA and RNA), polysaccharides (including heparin), as well as particles, such as viral particles, and cells. In one aspect, the bioactive agent has a molecular weight of about 10,000 or greater.

Classes of bioactive agents which can be incorporated into biodegradable implant (both the natural biodegradable matrix and/or the biodegradable microparticles) of this invention include, but are not limited to: ACE inhibitors, actin inhibitors, analgesics, anesthetics, anti-hypertensives, anti polymerases, antisecretory agents, anti-AIDS substances, antibiotics, anti-cancer substances, anti-cholinergics, anti-coagulants, anti-convulsants, anti-depressants, anti-emetics, antifungals, anti-glaucoma solutes, antihistamines, antihypertensive agents, anti-inflammatory agents (such as NSAIDs), anti metabolites, antimitotics, antioxidizing agents, anti-parasite and/or anti-Parkinson substances, antiproliferatives (including antiangiogenesis agents), anti-protozoal solutes, anti-psychotic substances, anti-pyretics, antiseptics, anti-spasmodics, antiviral agents, calcium channel blockers, cell response modifiers, chelators, chemotherapeutic agents, dopamine agonists, extracellular matrix components, fibrinolytic agents, free radical scavengers, growth hormone antagonists, hypnotics, immunosuppressive agents, immunotoxins, inhibitors of surface glycoprotein receptors, microtubule inhibitors, miotics, muscle contractants, muscle relaxants, neurotoxins, neurotransmitters, opioids, photodynamic therapy agents, prostaglandins, remodeling inhibitors, statins, steroids, thrombolytic agents, tranquilizers, vasodilators, and vasospasm inhibitors.

Antibiotics are art recognized and are substances which inhibit the growth of or kill microorganisms. Examples of antibiotics include penicillin, tetracycline, chloramphenicol, minocycline, doxycycline, vancomycin, bacitracin, kanamycin, neomycin, gentamycin, erythromycin, cephalosporins, geldanamycin, and analogs thereof. Examples of cephalosporins include cephalothin, cephapirin, cefazolin, cephalexin, cephradine, cefadroxil, cefamandole, cefoxitin, cefaclor, cefuroxime, cefonicid, ceforanide, cefotaxime, moxalactam, ceftizoxime, ceftriaxone, and cefoperazone.

Antiseptics are recognized as substances that prevent or arrest the growth or action of microorganisms, generally in a nonspecific fashion, e.g., by inhibiting their activity or destroying them. Examples of antiseptics include silver sulfadiazine, chlorhexidine, glutaraldehyde, peracetic acid, sodium hypochlorite, phenols, phenolic compounds, iodophor compounds, quaternary ammonium compounds, and chlorine compounds.

Anti-viral agents are substances capable of destroying or suppressing the replication of viruses. Examples of anti-viral agents include α-methyl-P-adamantane methylamine, hydroxy-ethoxymethylguanine, adamantanamine, 5-iodo-2′-deoxyuridine, trifluorothymidine, interferon, and adenine arabinoside.

Enzyme inhibitors are substances that inhibit an enzymatic reaction. Examples of enzyme inhibitors include edrophonium chloride, N-methylphysostigmine, neostigmine bromide, physostigmine sulfate, tacrine HCl, tacrine, 1-hydroxymaleate, iodotubercidin, p-bromotetramisole, 10-α-diethylaminopropionyl)-phenothiazine hydrochloride, calmidazolium chloride, hemicholinium-3,3,5-dinitrocatechol, diacylglycerol kinase inhibitor I, diacylglycerol kinase inhibitor II, 3-phenylpropargylamine, N-monomethyl-L-arginine acetate, carbidopa, 3-hydroxybenzylhydrazine HCl, hydralazine HCl, clorgyline HCl, deprenyl HCl, L(−), deprenyl HCl, D(+), hydroxylamine HCl, iproniazid phosphate, 6-MeO-tetrahydro-9H-pyrido-indole, nialamide, pargyline HCl, quinacrine HCl, semicarbazide HCl, tranylcypromine HCl, N,N-diethylaminoethyl-2,2-diphenylvalerate hydrochloride, 3-isobutyl-1-methylxanthine, papaverine HCl, indomethacin, 2-cyclooctyl-2-hydroxyethylamine hydrochloride, 2,3-dichloro-α-methylbenzylamine (DCMB), 8,9-dichloro-2,3,4,5-tetrahydro-1H-2-benzazepine hydrochloride, p-aminoglutethimide, p-aminoglutethimide tartrate, R(+), p-aminoglutethimide tartrate, S(−), 3-iodotyrosine, alpha-methyltyrosine, L(−) alpha-methyltyrosine, D L(−), cetazolamide, dichlorphenamide, 6-hydroxy-2-benzothiazolesulfonamide, and allopurinol.

Anti-pyretics are substances capable of relieving or reducing fever. Anti-inflammatory agents are substances capable of counteracting or suppressing inflammation. Examples of such agents include aspirin (salicylic acid), indomethacin, sodium indomethacin trihydrate, salicylamide, naproxen, colchicine, fenoprofen, sulindac, diflunisal, diclofenac, indoprofen and sodium salicylamide. Local anesthetics are substances that have an anesthetic effect in a localized region. Examples of such anesthetics include procaine, lidocaine, tetracaine and dibucaine.

Cell response modifiers are chemotactic factors such as platelet-derived growth factor (pDGF). Other chemotactic factors include neutrophil-activating protein, monocyte chemoattractant protein, macrophage-inflammatory protein, SIS (small inducible secreted) proteins, platelet factor, platelet basic protein, melanoma growth stimulating activity, epidermal growth factor, transforming growth factor (alpha), fibroblast growth factor, platelet-derived endothelial cell growth factor, insulin-like growth factor, nerve growth factor, and bone growth/cartilage-inducing factor (alpha and beta). Other cell response modifiers are the interleukins, interleukin inhibitors or interleukin receptors, including interleukin 1 through interleukin 10; interferons, including alpha, beta and gamma; hematopoietic factors, including erythropoietin, granulocyte colony stimulating factor, macrophage colony stimulating factor and granulocyte-macrophage colony stimulating factor; tumor necrosis factors, including alpha and beta; transforming growth factors (beta), including beta-1, beta-2, beta-3, inhibin, activin, and DNA that encodes for the production of any of these proteins.

Examples of statins include lovastatin, pravastatin, simvastatin, fluvastatin, atorvastatin, cerivastatin, rousvastatin, and superstatin.

Imaging agents are agents capable of imaging a desired site, e.g., tumor, in vivo, can also be included in the implant. Examples of imaging agents include substances having a label which is detectable in vivo, e.g., antibodies attached to fluorescent labels. The term antibody includes whole antibodies or fragments thereof.

Exemplary ligands or receptors include antibodies, antigens, avidin, streptavidin, biotin, heparin, type IV collagen, protein A, and protein G.

Exemplary antibiotics include antibiotic peptides.

The bioactive agent can provide antirestenotic effects, such as antiproliferative, anti-platelet, and/or antithrombotic effects. In some embodiments, the bioactive agent can include anti-inflammatory agents, immunosuppressive agents, cell attachment factors, receptors, ligands, growth factors, antibiotics, enzymes, nucleic acids, and the like. Compounds having antiproliferative effects include, for example, actinomycin D, angiopeptin, c-myc antisense, paclitaxel, taxane, and the like.

Representative examples of bioactive agents having antithrombotic effects include heparin, heparin derivatives, sodium heparin, low molecular weight heparin, hirudin, lysine, prostaglandins, argatroban, forskolin, vapiprost, prostacyclin and prostacyclin analogs, D-ph-pr-arg-chloromethylketone (synthetic antithrombin), dipyridamole, glycoprotein IIb/IIIa platelet membrane receptor antibody, coprotein IIb/IIIa platelet membrane receptor antibody, recombinant hirudin, thrombin inhibitor (such as commercially available from Biogen), chondroitin sulfate, modified dextran, albumin, streptokinase, tissue plasminogen activator (TPA), urokinase, nitric oxide inhibitors, and the like.

The bioactive agent can also be an inhibitor of the GPIIb-IIIa platelet receptor complex, which mediates platelet aggregation. GPIIb/IIa inhibitors can include monoclonal antibody Fab fragment c7E3, also know as abciximab (ReoPro™), and synthetic peptides or peptidomimetics such as eptifibatide (Integrilimm) or tirofiban (Agrastat™).

The bioactive agent can be an immunosuppressive agent, for example, cyclosporine, CD-34 antibody, everolimus, mycophenolic acid, sirolimus, tacrolimus, and the like.

Other exemplary therapeutic antibodies include trastuzumab (Herceptin™), a humanized anti-HER2 monoclonal antibody (moAb); alemtuzumab (Campath™), a humanized anti-CD52 moAb; gemtuzumab (Mylotarg™), a humanized anti-CD33 moAb; rituximab (Rituxamm), a chimeric anti-CD20 moAb; ibritumomab (Zevalin™), a murine moAb conjugated to a beta-emitting radioisotope; tositumomab (Bexxarm), a murine anti-CD20 moAb; edrecolomab (Panorex™), a murine anti-epithelial cell adhesion molecule moAb; cetuximab (Erbitux™), a chimeric anti-EGFR moAb; bevacizumab (Avastin™), a humanized anti-VEGF moAb, ranibizumab (Lucentis™), an anti-vascular endothelial growth factor mAb fragment, satumomab (OncoScint™) an anti-pancarcinoma antigen (Tag-72) mAb, pertuzumab (Omnitarg™) an anti-HER2 mAb, and daclizumab (Zenapax™) an anti IL-2 receptor mAb.

Additionally, the bioactive agent can be a surface adhesion molecule or cell-cell adhesion molecule. Exemplary cell adhesion molecules or attachment proteins (such as extracellular matrix proteins including fibronectin, laminin, collagen, elastin, vitronectin, tenascin, fibrinogen, thrombospondin, osteopontin, von Willibrand Factor, bone sialoprotein (and active domains thereof), or a hydrophilic polymer such as hyaluronic acid, chitosan or methyl cellulose, and other proteins, carbohydrates, and fatty acids. Exemplary cell-cell adhesion molecules include N-cadherin and P-cadherin and active domains thereof.

Exemplary growth factors include fibroblastic growth factors, epidermal growth factor, platelet-derived growth factors, transforming growth factors, vascular endothelial growth factor, bone morphogenic proteins and other bone growth factors, and neural growth factors.

The bioactive agent can be also be selected from mono-2-(carboxymethyl) hexadecanamidopoly(ethylene glycol)₂₀₀ mono-4-benzoylbenzyl ether, mono-3-carboxyheptadecanamidopoly(ethylene glycol)₂₀₀ mono-4-benzoylbenzyl ether, mono-2-(carboxymethyl)hexadecanamidotetra(ethylene glycol) mono-4-benzoylbenzyl ether, mono-3-carboxyheptadecanamidotetra(ethylene glycol) mono-4-benzoylbenzyl ether, N-[2-(4-benzoylbenzyloxy)ethyl]-2-(carboxymethyl)hexadecanamide, N-[2-(4-benzoylbenzyloxy)ethyl]-3-carboxyheptadecanamide, N-[12-(benzoylbenzyloxy)dodecyl]-2-(carboxymethyl)hexadecanamide, N-[12-(benzoylbenzyloxy)dodecyl]-3-carboxy-heptadecanamide, N-[3-(4-benzoylbenzamido) propyl]-2-(carboxymethyl)hexadecanamide, N-[3-(4-benzoylbenzamido) propyl]-3-carboxyheptadecanamide, N-(3-benzoylphenyl)-2-(carboxymethyl)hexadecanamide, N-(3-benzoylphenyl)-3-carboxyheptadecanamide, N-(4-benzoylphenyl)-2-(carboxymethyl)hexadecanamide, poly(ethylene glycol)₂₀₀ mono-15-carboxypentadecyl mono-4-benzoylbenzyl ether, and mono-15-carboxypentadecanamidopoly(ethylene glycol)₂₀₀ mono-4-benzoylbenzyl ether.

Additional examples of contemplated bioactive agents and/or bioactive agent include analogues of rapamycin (“rapalogs”), ABT-578 from Abbott, dexamethasone, betamethasone, vinblastine, vincristine, vinorelbine, poside, teniposide, daunorubicin, doxorubicin, idarubicin, anthracyclines, mitoxantrone, bleomycins, plicamycin (mithramycin), mitomycin, mechlorethamine, cyclophosphamide and its analogs, melphalan, chlorambucil, ethylenimines and methylmelamines, alkyl sulfonates-busulfan, nitrosoureas, carmustine (BCNU) and analogs, streptozocin, trazenes-dacarbazinine, methotrexate, fluorouracil, floxuridine, cytarabine, mercaptopurine, thioguanine, pentostatin, 2-chlorodeoxyadenosine, cisplatin, carboplatin, procarbazine, hydroxyurea, mitotane, estrogen, ticlopidine, clopidogrel, abciximab, breveldin, cortisol, cortisone, fludrocortisone, prednisone, prednisolone, 6U-methylprednisolone, triamcinolone, acetaminophen, etodalac, tolmetin, ketorolac, ibuprofen and derivatives, mefenamic acid, meclofenamic acid, piroxicam, tenoxicam, phenylbutazone, oxyphenthatrazone, nabumetone, auranofin, aurothioglucose, gold sodium thiomalate, azathioprine, mycophenolate mofetil; angiotensin receptor blockers; nitric oxide donors; and mTOR inhibitors.

Viral particles and viruses include those that may be therapeutically useful, such as those used for gene therapy, and also attenuated viral particles and viruses which can promote an immune response and generation of immunity. Useful viral particles include both natural and synthetic types. Viral particles include, but are not limited to, adenoviruses, baculoviruses, parvoviruses, herpesviruses, poxviruses, adeno-associated viruses, vaccinia viruses, and retroviruses.

Other bioactive agents that can be used for altering gene function include plasmids, phages, cosmids, episomes, and integratable DNA fragments, antisense oligonucleotides, antisense DNA and RNA, modified DNA and RNA, iRNA, ribozymes, siRNA, and shRNA.

Other bioactive agents include cells such as platelets, stem cells, T lymphocytes, B lymphocytes, acidophils, adipocytes, astrocytes, basophils, hepatocytes, neurons, cardiac muscle cells, chondrocytes, epithelial cells, dendrites, endrocrine cells, endothelial cells, eosinophils, erythrocytes, fibroblasts, follicular cells, ganglion cells, hepatocytes, endothelial cells, Leydig cells, parenchymal cells, lymphocytes, lysozyme-secreting cells, macrophages, mast cells, megakaryocytes, melanocytes, monocytes, myoid cells, neck nerve cells, neutrophils, oligodendrocytes, oocytes, osteoblasts, osteochondroclasts, osteoclasts, osteocytes, plasma cells, spermatocytes, reticulocytes, Schwann cells, Sertoli cells, skeletal muscle cells, and smooth muscle cells. Bioactive agents can also include genetically modified, recombinant, hybrid, mutated cells, and cells with other alterations.

Additives such as inorganic salts, BSA (bovine serum albumin), and inert organic compounds can be used to alter the profile of bioactive agent release, as known to those skilled in the art.

The concentration of the bioactive agent or agents dissolved or suspended in the mixture can range from about 0.01 to about 90 percent, by weight, based on the weight of the final composition.

The particular bioactive agent, or combination of bioactive agents, can be selected depending upon one or more of the following factors: the area of application of the ocular implant, the medical condition to be treated, the anticipated duration of treatment, characteristics of the implantation site, the number and type of bioactive agents to be utilized, and the like.

A comprehensive listing of bioactive agents can be found in The Merck Index, Thirteenth Edition, Merck & Co. (2001). Bioactive agents are commercially available from Sigma Aldrich Fine Chemicals, Milwaukee, Wis.

The bioactive agent can be present in the matrix of the ocular implant in particulate form. The particulates of bioactive agent can be from a powdered composition of the bioactive agent. In some cases, powders of bioactive agent can be formed from processes including precipitation and/or crystallization, and spray drying. Small particulates, such as microparticles, can also be formed by processes such as micronizing, milling, grinding, crushing, and chopping.

In some aspects of the invention, a microparticle is used to deliver the bioactive agent from the natural biodegradable polysaccharide-based ocular implant. The microparticles of bioactive agent can comprise any three-dimensional structure that can be immobilized in the matrix formed by the biodegradable polysaccharide.

The term “microparticle” is intended to reflect that the three-dimensional structure is very small but not limited to a particular size range, or not limited to a structure that has a particular shape. According to the invention, microparticles typically have a size in the range of 5 nm to 100 μm in diameter. In some embodiments, the microparticles have a size in the range of 100 nm to 20 μm in diameter, and even more preferable in the range of 400 nm to 20 μm in diameter.

In some aspects, the ocular implants can have two, or more than two, different bioactive agents present in the matrix of biodegradable polysaccharides. The bioactive agents may be mutually incompatible in a particular environment, for example, as hydrophobic and hydrophilic drugs are incompatible in either a polar or non-polar solvent. Different bioactive agents may also demonstrate incompatibility based on protic/aprotic solvents or ionic/non-ionic solvents. For example, the invention allows for the preparation of one set of microparticles containing a hydrophobic drug and the preparation of another set of microparticles containing a hydrophilic drug; the mixing of the two different sets of microparticles into a polymeric material used to form the matrix; and then forming an ocular implant. Both hydrophobic and hydrophilic drugs can be released from the ocular implant at the same time or the natural biodegradable polysaccharide matrix can be altered so that one bioactive agent is released at a different rate or time than the other one.

Implants of the invention are typically designed to minimize interference with the functions of the eye and discomfort and damage to the eye. In some embodiments, the implant is rod-like or filament-like in shape. In some embodiments, the implant may have a distal end that is beveled, tapered, or sharpened. Alternatively, the implant may have a distal end that is blunt or rounded.

In some embodiments, the implant has a total diameter that is no greater than about 1000 μm, in other embodiments no greater than about 900 μm, in other embodiments no greater than about 800 μm, in other embodiments no greater than about 700 μm, in other embodiments no greater than about 600 μm, in other embodiments no greater than about 500 μm, in other embodiments no greater than about 400 μm, in other embodiments no greater than about 300 μm, in other embodiments no greater than about 200 μm, in other embodiments no greater than about 100 μm, in other embodiments no greater than about 50 μm. In some embodiments, the total diameter of the implant ranges from about 200 μm to about 500 μm.

In some embodiments, the implants of the invention have a length that is no greater than about 5 mm, in other embodiments no greater than about 4.5 mm, in other embodiments no greater than about 4 mm, in other embodiments no greater than about 3.5 mm, in other embodiments no greater than about 3.0 mm, in other embodiments no greater than about 2.9 mm, in other embodiments no greater than about 2.8 mm, in other embodiments no greater than about 2.7 mm, in other embodiments no greater than about 2.6 mm, in other embodiments no greater than about 2.5 mm, in other embodiments no greater than about 2.4 mm, in other embodiments no greater than about 2.3 mm, in other embodiments no greater than about 2.2 mm, in other embodiments no greater than about 2.1 mm, in other embodiments no greater than about 2 mm. In some embodiments, the length of the implant ranges from about 2.25 mm to about 2.75 mm.

In some aspects of the invention the natural biodegradable polymer is used to form the body member of an ocular implant, wherein the body member has a dry weight of about 6 mg or less. In some aspects the body member has a dry weight of about 2.5 mg or less. In some aspects the body member has a dry weight of about 2.3 mg or less. In some aspects the body member has a dry weight of about 2.0 mg or less. In some aspects the body member has a dry weight of about 1.8 mg or less. In some aspects the body member has a dry weight of about 1.5 mg or less.

The ocular implants can have a defined structure and can be formed by any suitable process, including molding, extruding, shaping, cutting, casting, and the like.

A molding process exemplifies a process for forming the ocular implants of the present invention. A composition including acrylated maltodextrin, a high molecular weight bioactive agent (such as a polypeptide), and a photoactivatable polymerization initiator is prepared. The composition is disposed in a plastic mold that allows UV light to pass through the mold material and then sealed. The mold can be plastic tubing having inner dimensions in the desired size and shape of the ocular implant. The mold is then treated with UV light to initiate polymerization and matrix formation, thereby forming the implant. The mold is then unsealed and the implant is removed.

In some aspects of the invention, the natural biodegradable polysaccharide compositions can be used to form an ocular implant with an optically clear matrix. For example, maltodextrin and polyalditol can be formed into optically clear matrices using either redox or photoinitiation. Factors that can affect the ability of the formed matrix to be optically clear include the water solubility of the macromers utilized to form the matrix, and/or transparency of the initiating reagents. It will be readily appreciated that optically clear matrices formed in accordance with the invention can provide significant benefits, since such matrices can form implants that will not adversely impact the patient's vision (e.g., by creating blind spots by virtue of interference from the implant material). In turn, this can allow more flexibility as to the size and/or location of an ocular implant located within the interior of the eye.

The implant can also be dehydrated, or de-liquefied, prior to implantation in a subject. Typically, the composition includes a certain amount of water, or a polar liquid, which remains in the matrix following its formation. The matrix can be air-dried or vacuum dried to remove some of or most all of the liquid present in the matrix. Upon dehydration, the matrix may also shrink somewhat.

The implant in a substantially or fully dehydrated form can have a certain amount of components, as conveyed as a percentage weight of the implant. In some aspects, the percentage of biodegradable polymer by total weight of the implant is about 80 wt % or greater, about 85 wt % or greater, about 87.5 wt % or greater, about 90 wt % or greater, about 92.5 wt % or greater, or about 95 wt % or greater.

In the partially or fully dehydrated implant, and in some aspects, the percentage of bioactive agent (or combination of bioactive agents) by total weight of the implant is up to about 15 wt %, up to about 12.5 wt %, such as in the range of about 0.1 wt % to about 15 wt %, in the range of about 2.5 wt % to about 12.5 wt %, or in the range of about 5 wt % to about 11 wt %.

The ocular implant can be sterilized before insertion into the eye. In some aspects the ocular implant can be contacted with an aqueous sterilization solution.

The implant can be provided to an individual that performs the implantation procedure in a partially dehydrated or fully dehydrated form. After the implant has been inserted into the inner eye, such as in the vitreous, it can undergo partial or full rehydration. The rehydration may cause some swelling of the implant, and an increase in size may be observed.

In accordance with the invention, the biodegradable ocular implant can be implanted into a portion of the eye using any suitable method. Typically, the implant is administered by using an insertion instrument to provide the implant to a target site within the eye. The term “implantation site” refers to the site within a patient's body at which the ocular implant is located during a treatment course according to the invention.

The ocular can be placed at an implantation site within the eye tissues. Suitable ocular implants can perform a function and/or provide bioactive agent to any desired area of the eye. In some aspects, the ocular implant can be utilized to deliver bioactive agent to an anterior segment of the eye (in front of the lens), and/or a posterior segment of the eye (behind the lens). Suitable ocular implant can also be utilized to provide bioactive agent to tissues in proximity to the eye, when desired.

Ocular implants configured for placement at an internal site of the eye can reside within any desired area of the eye. In some aspects, the ophthalmic article can be configured for placement at an intraocular site, such as the vitreous or subretinal space.

As mentioned, the vitreous chamber is the largest chamber of the eye and contains the vitreous humor or vitreous. Generally speaking, the vitreous is bound interiorly by the lens, posterior lens zonules and ciliary body, and posteriorly by the retinal cup. The vitreous is a transparent, viscoelastic gel that is 98% water and has a viscosity of about 2-4 times that of water. The main constituents of the vitreous are hyaluronic acid (HA) molecules and type II collagen fibers, which entrap the HA molecules. The viscosity is typically dependent on the concentration of HA within the vitreous. The vitreous is traditionally regarded as consisting of two portions: a cortical zone, characterized by more densely arranged collagen fibrils, and a more liquid central vitreous.

Therefore, in some aspects, the invention provides method for placing an ocular implant at a site within the body, the site comprising a gel-like material, such as viscoelastic gel.

In many aspects of the invention, the ocular implant is placed in the vitreous. In some aspects, the ocular implant can be delivered through the scleral tissue (trans-scleral injection). Typically, intravitreal delivery will be accomplished by using an insertion instrument utilizing a 25 to 30-gauge needle (or smaller) having a length of about 0.5 inches to about 0.62 inches.

This methodology also yields a technique that can be implemented in an outpatient clinic setting. According to this embodiment, a insertion instrument or device is provided (e.g., a cannula or syringe), a portion of which is configured and arranged such that when the instrument is inserted into the eye, the opening formed in the sclera to receive the instrument is small enough so as to not require sutures to seal or close the opening in the sclera. In other words, the opening is small enough that the wound or opening is self-sealing, thereby preventing the vitreous humor from leaking out of the eye.

In addition, the step of inserting can further include inserting the insertable portion of the insertion instrument or device transconjunctivally so the operable end thereof is within the vitreous. In this regard, transconjunctival shall be understood to mean that the instrument's operable end is inserted through both the conjunctiva and through the sclera into the vitreous. More particularly, inserting the insertable portion that forms an opening in the sclera and the conjunctiva that is small enough so as to not require sutures or the like to seal or close the opening in the sclera. In conventional surgical techniques for the posterior segment of the eye, the conjunctiva is routinely dissected to expose the sclera, whereas according to the methodology of this embodiment, the conjunctiva need not be dissected or pulled back.

Consequently, when the instrument is removed from the eye, the surgeon does not have to seal or close the opening in the sclera with sutures to prevent leaking of the aqueous humor, since such an opening or wound in the sclera is self-sealing. In addition, with the transconjunctival approach, the surgeon does not have to reattach the dissected conjunctiva. These features can further simplify the surgical procedure, as well as reduce (if not eliminate) suturing required under the surgical procedure.

It will be understood that the inventive methods do not require dissection of the conjunctiva. However, if such additional step is desired in a particular treatment, such conjunctival dissection could be performed.

The insertion procedure can be performed without vitrectomy and results in a self-sealing sclerotomy, eliminating the need for sutures and minimizing risk of infection. In some aspects, the small sclerotomy is leakage-free, thereby reducing risk of leakage of vitreous from the implantation site. Advantageously, the inventive methods can be performed as an office-based procedure.

In some aspects, the ocular implant in placed at a subretinal area within the eye. An insertion instrument can be advanced transconjunctivally and trans-retinally, to reach the subretinal space within the eye to deliver the implant. Once the tip of the instrument has reached the subretinal space, a limited or localized retinal detachment (e.g., a bleb detachment) can be formed using any of a number of devices and/or techniques known to those skilled in the art, thereby defining or forming a subretinal space. The implant can then be placed in the subretinal space formed by the retinal detachment. The limited or local dome-shaped subretinal detachment is created in such a fashion that the detachment itself generally does not have an appreciable or noticeable long-term effect on the vision of the patient.

In some cases, a grasping member (such as forceps) can be used to locate (for example, by pulling) the ocular implant at the desired implantation site. The ocular implant can then reside at the implantation site during a treatment course.

In some aspects, the invention provides a method for delivering a bioactive agent from ocular implant by exposing the ocular implant to an enzyme that causes the degradation of the implant. In performing this method ocular implant is provided to a subject. The ocular implant is then exposed to a carbohydrase that can promote the degradation of the ocular implant.

The carbohydrase that contacts the ocular implant can specifically degrade the natural biodegradable polysaccharide causing release of the bioactive agent. Examples of carbohydrases that can specifically degrade natural biodegradable polysaccharide implants include α-amylases, such as salivary and pancreatic α-amylases; disaccharidases, such as maltase, lactase and sucrase; trisaccharidases; and glucoamylase (amyloglucosidase).

Serum concentrations for amylase are estimated to be in the range of about 50-100 Upper liter, and vitreal concentrations also fall within this range (Varela, R. A., and Bossart, G. D. (2005) J Am Vet Med Assoc 226:88-92).

In some aspects, the carbohydrase can be administered to a subject to increase the local concentration, for example in the serum or the tissue surrounding the implanted device, so that the carbohydrase may promote the degradation of the implant. Exemplary routes for introducing a carbohydrase include local injection, intravenous (IV) routes, and the like. Alternatively, degradation can be promoted by indirectly increasing the concentration of a carbohydrase in the vicinity of the implant, for example, by a dietary process, or by ingesting or administering a compound that increases the systemic levels of a carbohydrase. In some cases a carbohydrase can be delivered to a portion of the eye, by, for example, injection.

In other cases, the carbohydrase can be provided on a portion of the ocular implant. For example the carbohydrase may be eluted from a portion of the ocular implant. In this aspect, as the carbohydrase is released it locally acts upon the ocular implant to cause its degradation and promote the release of the bioactive agent. Alternatively, the carbohydrase can be present in a particle in one or more portions the ocular implant. As the carbohydrase is released from the particle, it causes degradation and promotes the release of the bioactive agent.

The invention will be further described with reference to the following non-limiting Examples. It will be apparent to those skilled in the art that many changes can be made in the embodiments described without departing from the scope of the present invention. Thus the scope of the present invention should not be limited to the embodiments described in this application, but only by embodiments described by the language of the claims and the equivalents of those embodiments. Unless otherwise indicated, all percentages are by weight.

### EXAMPLE 1

### Synthesis of Acrylated-Amylose

Amylose having polymerizable vinyl groups was prepared by mixing 0.75 g of amylose (A0512; Aldrich) with 100 mL of methylsulfoxide (JT Baker) in a 250 mL amber vial, with stirring. After one hour, 2 mL of triethylamine (TEA; Aldrich) was added and the mixture was allowed to stir for 5 minutes at room temperature. Subsequently, 2 mL of glycidyl acrylate (Polysciences) was added and the amylose and glycidyl acrylate were allowed to react by stirring overnight at room temperature. The mixture containing the amylose-glycidyl acrylate reaction product was dialyzed for 3 days against DI water using continuous flow dialysis. The resultant acrylated-amylose (0.50 g; 71.4% yield) was then lyophilized and stored desiccated at room temperature with protection from light.

### EXAMPLE 2

### Synthesis of MTA-PAAm

A polymerization initiator was prepared by copolymerizing a methacrylamide having a photoreactive group with acrylamide.

A methacrylamide-oxothioxanthene monomer (N-[3-(7-Methyl-9-oxothioxanthene-3-carboxamido) propyl]methacrylamide (MTA-APMA)) was first prepared. N-(3-aminopropyl)methacrylamide hydrochloride (APMA), 4.53 g (25.4 mmol), prepared as described in U.S. Pat. No. 5,858,653, Example 2, was suspended in 100 mL of anhydrous chloroform in a 250 mL round bottom flask equipped with a drying tube. 7-methyl-9-oxothioxanthene-3-carboxylic acid (MTA) was prepared as described in U.S. Pat. No. 4,506,083, Example D. MTA-chloride (MTA-Cl) was made as described in U.S. Pat. No. 6,007,833, Example 1. After cooling the slurry in an ice bath, MTA-Cl (7.69 g; 26.6 mmol) was added as a solid with stirring to the APMA-chloroform suspension. A solution of 7.42 mL (53.2 mmol) of TEA in 20 mL of chloroform was then added over a 1.5 hour time period, followed by a slow warming to room temperature. The mixture was allowed to stir 16 hours at room temperature under a drying tube. After this time, the reaction was washed with 0.1 N HCl and the solvent was removed under vacuum after adding a small amount of phenothiazine as an inhibitor. The resulting product was recrystallized from tetrahydrofuran (THF)/toluene (3/1) and gave 8.87 g (88.7% yield) of product after air drying. The structure of MTA-APMA was confirmed by NMR analysis.

MTA-APMA was then copolymerized with acrylamide in DMSO in the presence of 2-mercaptoethanol (a chain transfer agent), N,N,N′,N′-tetramethyl-ethylenediamine (a co-catalyst), and 2,2′-azobis(2-methyl-propionitrile) (a free radical initiator) at room temperature. The solution was sparged with nitrogen for 20 minutes, sealed tightly, and incubated at 55° C. for 20 hours. The solution was dialyzed for 3 days against DI water using continuous flow dialysis. The resultant MTA-PAAm was lyophilized, stored desiccated, and protected from light at room temperature.

### EXAMPLE 3

### Preparation of 4-bromomethylbenzophenone (BMBP)

4-Methylbenzophenone (750 g; 3.82 moles) was added to a 5 liter Morton flask equipped with an overhead stirrer and dissolved in 2850 mL of benzene. The solution was then heated to reflux, followed by the dropwise addition of 610 g (3.82 moles) of bromine in 330 mL of benzene. The addition rate was approximately 1.5 mL/min and the flask was illuminated with a 90 watt (90 joule/sec) halogen spotlight to initiate the reaction. A timer was used with the lamp to provide a 10% duty cycle (on 5 seconds, off 40 seconds), followed in one hour by a 20% duty cycle (on 10 seconds, off 40 seconds). At the end of the addition, the product was analyzed by gas chromatography and was found to contain 71% of the desired 4-bromomethylbenzophenone, 8% of the dibromo product, and 20% unreacted 4-methylbenzophenone. After cooling, the reaction mixture was washed with 10 g of sodium bisulfite in 100 mL of water, followed by washing with 3×200 mL of water. The product was dried over sodium sulfate and recrystallized twice from 1:3 toluene:hexane. After drying under vacuum, 635 g of 4-bromomethylbenzophenone was isolated, providing a yield of 60%, having a melting point of 112° C.-114° C. Nuclear magnetic resonance (“NMR”) analysis (¹H NMR (CDCl₃)) was consistent with the desired product: aromatic protons 7.20-7.80 (m, 9H) and methylene protons 4.48 (s, 2H). All chemical shift values are in ppm downfield from a tetramethylsilane internal standard.

### EXAMPLE 4

### Preparation of ethylenebis(4-benzoylbenzyldimethylammonium)dibromide

N,N,N′,N′-Tetramethylethylenediamine (6 g; 51.7 mmol) was dissolved in 225 mL of chloroform with stirring. BMBP (29.15 g; 106.0 mmol), as described in Example 3, was added as a solid and the reaction mixture was stirred at room temperature for 72 hours. After this time, the resulting solid was isolated by filtration and the white solid was rinsed with cold chloroform. The residual solvent was removed under vacuum and 34.4 g of solid was isolated for a 99.7% yield, melting point 218° C.-220° C. Analysis on an NMR spectrometer was consistent with the desired product: ¹H NMR (DMSO-d₆) aromatic protons 7.20-7.80 (m, 18H), benzylic methylenes 4.80 (br. s, 4H), amine methylenes 4.15 (br. s, 4H), and methyls 3.15 (br. s, 12H).

### EXAMPLE 5

### Preparation of 1-(6-oxo-6-hydroxyhexyl)maleimide (Mal-EACA)

A maleimide functional acid was prepared in the following manner, and was used in Example 6. EACA (6-aminocaproic acid), (100 g; 0.762 moles), was dissolved in 300 mL of acetic acid in a three-neck, three liter flask equipped with an overhead stirrer and drying tube. Maleic anhydride, (78.5 g; 0.801 moles), was dissolved in 200 mL of acetic acid and added to the EACA solution. The mixture was stirred one hour while heating on a boiling water bath, resulting in the formation of a white solid. After cooling overnight at room temperature, the solid was collected by filtration and rinsed two times with 50 mL of hexane each rinse. After drying, the yield of the (z)-4-oxo-5-aza-undec-2-endioic acid (Compound 1) was in the range of 158-165 g (90-95%) with a melting point of 160-165° C. Analysis on an NMR spectrometer was consistent with the desired product: ¹H NMR (DMSO-d₆, 400 MHz) δ 6.41, 6.24 (d, 2H, J=12.6 Hz; vinyl protons), 3.6-3.2 (b, 1H; amide proton), 3.20-3.14 (m, 2H: methylene adjacent to nitrogen), 2.20 (t, 2H, J=7.3; methylene adjacent to carbonyl), 1.53-1.44 (m, 4H; methylenes adjacent to the central methylene), and 1.32-1.26 (m, 2H; the central methylene).

(z)-4-oxo-5-aza-undec-2-endioic acid, (160 g; 0.698 moles), zinc chloride, 280 g (2.05 moles), and phenothiazine, 0.15 g were added to a two liter round bottom flask fitted with an overhead stirrer, condenser, thermocouple, addition funnel, an inert gas inlet, and heating mantle. Chloroform (CHCl₃), 320 mL was added to the 2 liter reaction flask, and stirring of the mixture was started. Triethylamine (480 mL; 348 g, 3.44 moles (TEA)) was added over one hour. Chlorotrimethyl silane (600 mL; 510 g, 4.69 moles) was then added over two hours. The reaction was brought to reflux and was refluxed overnight (˜16 hours). The reaction was cooled and added to a mixture of CHCl₃ (500 mL), water (1.0 liters), ice (300 g), and 12 N hydrochloric acid (240 mL) in a 20 liter container over 15 minutes. After 15 minutes of stirring, the aqueous layer was tested to make sure the pH was less than 5. The organic layer was separated, and the aqueous layer was extracted three times with CHCl₃ (700 mL) each extraction. The organic layers were combined and evaporated on a rotary evaporator. The residue was then placed in a 20 liter container. A solution of sodium bicarbonate (192 g) in water (2.4 liters) was added to the residue. The bicarbonate solution was stirred until the solids were dissolved. The bicarbonate solution was treated with a solution of hydrochloric acid, (26 liters of 1.1 N) over 5 minutes to a pH of below 2. The acidified mixture was then extracted with two portions of CHCl₃, (1.2 liters and 0.8 liters) each extraction. The combined extracts were dried over sodium sulfate and evaporated. The residue was recrystallized from toluene and hexane. The crystalline product was then isolated by filtration and dried which produced 85.6 g of white N-(6-oxo-6-hydroxyhexyl)maleimide (Mal-EACA; Compound 2). Analysis on an NMR spectrometer was consistent with the desired product: ¹H NMR (CDCl₃, 400 MHz) δ 6.72 (s, 2H; maleimide protons), 3.52 (t, 2H, J=7.2 Hz; methylene next to maleimide), 2.35 (t, 2H, J=7.4; methylene next to carbonyl), 1.69-1.57 (m, 4H; methylenes adjacent to central methylene), and 1.39-1.30 (m, 2H; the central methylene). The product had a DSC (differential scanning calorimator) melting point peak at 89.9° C.

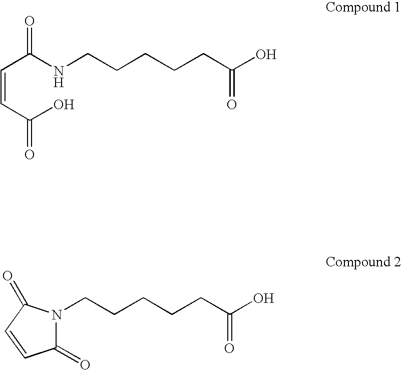

### EXAMPLE 6

### Preparation of N-(5-isocvanatopentyl)maleimide (Mal-C5-NCO)

Mal-EACA from Example 5 (5.0 g; 23.5 mmole) and CHCl₃ (25 mL) were placed in a 100 mL round bottom flask and stirred using a magnetic bar with cooling in an ice bath. Oxalyl chloride (10.3 mL; ˜15 g; 118 mmole) was added and the reaction was brought to room temperature with stirring overnight. The volatiles were removed on a rotary evaporator, and the residue was azetroped with three times with 10 mL CHCl₃ each time. The intermediate Mal-EAC-Cl [N-(6-oxo-6-chlorohexyl)maleimide] (Compound 3) was dissolved in acetone (10 mL) and added to a cold (ice bath) stirred solution of sodium azide (2.23 g; 34.3 mmole) in water (10 mL). The mixture was stirred one hour using an ice bath. The organic layer was set aside in an ice bath, and the aqueous layer was extracted three times with 10 mL CHCl₃. All operations of the acylazide were done at ice bath temperatures. The combined organic solutions of the azide reaction were dried for an hour over anhydrous sodium sulfate. The N-(6-oxo-6-azidohexyl)maleimide (Compound 4) solution was further dried by gentle swirling over molecular sieves over night. The cold azide solution was filtered and added to refluxing CHCl₃, 5 mL over a 10 minute period. The azide solution was refluxed for 2 hours. The weight of Mal-C5-NCO (Compound 5) solution obtained was 55.5 g, which was protected from moisture. A sample of the isocyanate solution, 136 mg was evaporated and treated with DBB (1,4-dibromobenzene), 7.54 mg and chloroform-d, 0.9 mL: ¹H NMR (CDCl₃, 400 MHz) δ 6.72 (s, 2H), 3.55 (t, 2H, J=7.2 Hz), 3.32 (t, 2H, J=6.6 Hz), 1.70-1.59 (m, 4H), 1.44-1.35 (m, 2H). The NMR spectra was consistent with desired product. The DBB internal standard 6 at 7.38 (integral value was 2.0, 4H; per mole of product) was used to estimate the moles of Mal-C5-NCO in solution. The calculated amount of product in solution was 23.2 mmole for a yield of 98% of theory. NCO reagent (concentration was 0.42 mmole/g) was used to prepare a macromer in Example 12.

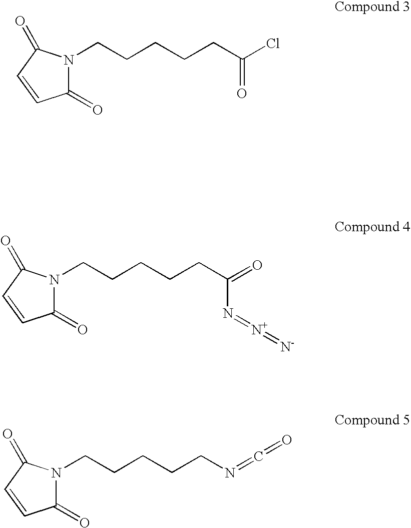

### EXAMPLE 7

### Preparation of 3-(acryloyloxy)propanoic acid (2-carboxyethyl acrylate; CEA)

Acrylic acid (100 g; 1.39 mole) and phenothiazine (0.1 g) were placed in a 500 mL round bottom flask. The reaction was stirred at 92° C. for 14 hours. The excess acrylic acid was removed on a rotary evaporator at 25° C. using a mechanical vacuum pump. The amount of residue obtained was 51.3 g. The CEA (Compound 6) was used in Example 7 without purification.

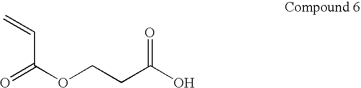

### EXAMPLE 8

### Preparation of 3-chloro-3-oxopropyl acrylate (CEA-Cl)

CEA from Example 7 (51 g; ˜0.35 mole) and dimethyl formamide (DMF; 0.2 mL; 0.26 mmole) were dissolved in CH₂Cl₃ (100 mL). The CEA solution was added slowly (over 2 hours) to a stirred solution of oxalyl chloride (53 mL; 0.61 mole), DMF (0.2 mL; 2.6 mmole), anthraquinone (0.5 g; 2.4 mmole), phenothiazine (0.1 g, 0.5 mmole), and CH₂Cl₃ (75 mL) in a 500 mL round bottom flask in an ice bath at 200 mm pressure. A dry ice condenser was used to retain the CH₂Cl₃ in the reaction flask. After the addition was complete the reaction was stirred at room temperature overnight. The weight of reaction solution was 369 g. A sample of the CEA-Cl (Compound 7) reaction solution (124 mg) was treated with 1,4-dibromobenzene (DBB, 6.85 mg) evaporated and dissolved in CDCl₃: ¹H NMR (CDCl₃, 400 MHz) δ 7.38 (s, 4H; DBB internal std.), 6.45 (d, 1H, J=17.4 Hz), 6.13 (dd, 1H, J=17.4, 10.4 Hz), 5.90 (d, 1H, J=10.4 Hz), 4.47 (t, 2H, J=5.9 Hz), 3.28 (t, 2H, J=5.9). The spectra was consistent with the desired product. There was 0.394 mole DBB for 1.0 mole CEA-Cl by integration, which gave a calculated yield of 61%. Commercially available CEA (426 g; Aldrich) was reacted with oxalyl chloride (532 mL) in a procedure similar to the one listed above. The residue CEA-Cl (490 g) was distilled using an oil bath at 140° C. at a pressure of 18 mm Hg. The distillate temperature reached 98° C. and 150 g of distillate was collected. The distillate was redistilled at 18 mm Hg at a maximum bath temperature of 120° C. The temperature range for the distillate was 30° C. to 70° C. which gave 11 g of material. The distillate appeared to be 3-chloro-3-oxopropyl 3-chloropropanoate. The residue of the second distillation (125 g; 26% of theory) was used in Example 9.

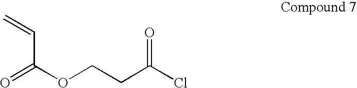

### EXAMPLE 9

### Preparation of 3-azido-3-oxopropyl acrylate (CEA-N3)

CEA-Cl from Example 7 (109.2 g; 0.671 mole) was dissolved in acetone (135 mL). Sodium azide (57.2 g; 0.806 mole) was dissolved in water (135 mL) and chilled. The CEA-Cl solution was then added to the chilled azide solution with vigorous stirring in an ice bath for 1.5 hours. The reaction mixture was extracted two times with 150 mL of CHCl₃ each extraction. The CHCl₃ solution was passed through a silica gel column 40 mm in diameter by 127 mm. The 3-azido-3-oxopropyl acrylate (Compound 8) solution was gently agitated over dried molecular sieves at 4° C. overnight. The dried solution was used in Example 10 without purification.

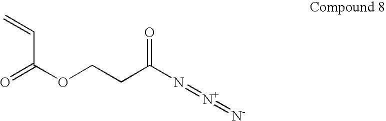

### EXAMPLE 10

### Preparation of 2-isocyanatoethyl Acrylate (EA-NCO)

The dried azide solution (from Example 9) was slowly added to refluxing CHCl₃, 75 mL. After the addition was completed, refluxing was continued 2 hours. The EA-NCO (Compound 9) solution (594.3 g) was protected from moisture. A sample of the EA-NCO solution (283.4 mg) was mixed with DBB (8.6 mg) and evaporated. The residue was dissolved in CDCl₃: ¹H NMR (CDCl₃, 400 MHz) δ 7.38 (s, 4H; DBB internal std.), 6.50 (d, 1H, J=17.3 Hz), 6.19 (dd, 1H, J=17.3, 10.5 Hz), 5.93 (d, 1H, J=10.5 Hz), 4.32 (t, 2H, J=5.3 Hz), 3.59 (t, 2H, J=5.3). The spectra was consistent with the desired EA-NCO. There was 0.165 mole DBB for 1.0 mole EA-NCO by integration, which gave a calculated concentration of 110 mg EA-NCO/g of solution. The EA-NCO solution was used to prepare a macromer in Example 11.

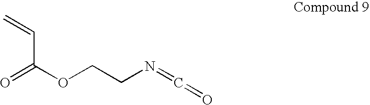

### EXAMPLE 11

### Preparation of Maltodextrin-Acrylate Macromer (MD-Acrylate)

Maltodextrin (MD; Aldrich; 9.64 g; ˜3.21 mmole; DE (Dextrose Equivalent): 4.0-7.0) was dissolved in dimethylsulfoxide (DMSO) 60 mL. The size of the maltodextrin was calculated to be in the range of 2,000 Da-4,000 Da. A solution of EA-NCO from Example 10 (24.73 g; 19.3 mmole) was evaporated and dissolved in dried DMSO (7.5 mL). The two DMSO solutions were mixed and heated to 55° C. overnight. The DMSO solution was placed in dialysis tubing (1000 MWCO, 45 mm flat width×50 cm long) and dialyzed against water for 3 days. The macromer solution was filtered and lyophilized to give 7.91 g white solid. A sample of the macromer (49 mg), and DBB (4.84 mg) was dissolved in 0.8 mL DMSO-d₆: ¹H NMR (DMSO-d₆, 400 MHz) δ 7.38 (s, 4H; internal std. integral value of 2.7815), 6.50, 6.19, and 5.93 (doublets, 3H; vinyl protons integral value of 3.0696). The calculated acrylate load of macromer was 0.616 μmoles/mg of polymer.

### EXAMPLE 12

### Preparation of Maltodextrin-Maleimide Macromer (MD-Mal)

A procedure similar to Example 11 was used to make the MD-Mal macromer. A solution of Mal-C5-NCO from Example 6 (0.412 g; 1.98 mmole) was evaporated and dissolved in dried DMSO (2 mL). MD (0.991 g; 0.33 mmole) was dissolved in DMSO (5 mL). The DMSO solutions were combined and stirred at 55° C. for 16 hours. Dialysis and lyophilization gave 0.566 g product. A sample of the macromer (44 mg), and DBB (2.74 mg) was dissolved in 00.8 mL DMSO-d₆: ¹H NMR (DMSO-d₆, 400 MHz) δ 7.38 (s, 4H; internal std. integral value of 2.3832), 6.9 (s, 2H; Maleimide protons integral value of 1.000). The calculated acrylate load of macromer was 0.222 μmoles/mg of polymer. The macromer was tested for its ability to make a matrix (see Example 15)

### EXAMPLE 13

### Formation of Maltodextrin-acrylate biodegradable matrix using MTA-PAAm

250 mg of MD-Acrylate as prepared in Example 11 was placed in an 8 mL amber vial. To the MD-Acrylate was added 3 mg of MTA-PAAm (lyophilized), 2 μL of 2-NVP, and 1 mL of 1× phosphate-buffered saline (1×PBS), providing a composition having MD-Acrylate at a 20% solids content. The reagents were then mixed for one hour on a shaker at 37° C. The mixture in an amount of 50 μL was placed onto a glass slide and illuminated for 40 seconds with an EFOS 100 SS illumination system equipped with a 400-500 nm filter. After illumination the polymer was found to form a semi-firm gel having elastomeric properties.

### EXAMPLE 14

### Formation of MD-Acrylate Biodegradable Matrix Using Camphorquinone

250 mg of MD-acrylate as prepared in Example 11 was placed in an 8 mL amber vial. To the MD-Acrylate was added 14 mg of camphorquinone-10-sulfonic acid hydrate (Toronto Research Chemicals, Inc.), 3 μL of 2-NVP, and 1 mL of distilled water. The reagents were then mixed for one hour on a shaker at 37° C. The mixture in an amount of 50 μL was placed onto a glass slide and illuminated for 40 seconds with a SmartliteIQ™ LED curing light (Dentsply Caulk). After illumination the polymer was found to form a semi-firm gel having with elastomeric properties.

### EXAMPLE 15

### Formation of MD-Mal Biodegradable Matrix Using MTA-PAAm

250 mg of MD-Mal as prepared in Example 12 was placed in an 8 mL amber vial. To the MD-Mal was added 3 mg of MTA-PAAm (lyophilized), 2 μL of 2-NVP, and 1 mL of 1× phosphate-buffered saline (1×PBS). The reagents were then mixed for one hour on a shaker at 37° C. The mixture in an amount of 50 μL was placed onto a glass slide and illuminated for 40 seconds with an EFOS 100 SS illumination system equipped with a 400-500 nm filter. After illumination the polymer was found to form a semi-firm gel having elastomeric properties.

### EXAMPLE 16

### Bioactive Agent Incorporation/Release from a MD-Acrylate Matrix

500 mg of MD-Acrylate as prepared in Example 11 was placed in an 8 mL amber vial. To the MD-Acrylate was added 3 mg of MTA-PAAm (lyophilized), 2 μL of 2-NVP, and 1 mL of 1× phosphate-buffered saline (1×PBS). The reagents were then mixed for one hour on a shaker at 37° C. To this mixture was added either 5 mg 70 kD FITC-Dextran or 5 mg 10 kD FITC-Dextran (Sigma) and vortexed for 30 seconds. The mixture in an amount of 200 μL was placed into a Teflon well plate (8 mm diameter, 4 mm deep) and illuminated for 40 seconds with an EFOS 100 SS illumination system equipped with a 400-500 nm filter. The formed matrix was loose, and not as well crosslinked as the formed MD-acrylate matrix in Example 15. After illumination, the matrix was transferred to a 12 well plate (Falcon) and placed in a well containing 0.6 mL PBS. At daily intervals for 6 days, 150 μL of PBS was removed from each well and placed into a 96 well plate. The remaining 850 μL were removed from the samples, and replaced with 1 mL fresh PBS. The 96 well plate was analyzed for FITC-Dextran on a spectrophotometer (Shimadzu) at 490 absorbance. Results showed that at least 70% of the detectable 1 Okd or 70 kD FITC-Dextran was released from the matrix after 2 days. Visual observation showed that an unquantified amount of 10 kD or 70 kD FITC-Dextran remained within the matrix after 6 days.

### EXAMPLE 17

### Polyalditol-acrylate synthesis

Polyalditol (PA; GPC; 9.64 g; ˜3.21 mmole) was dissolved in dimethylsulfoxide (DMSO) 60 mL. The size of the polyalditol was calculated to be in the range of 2,000 Da-4,000 Da. A solution of EA-NCO from Example 10 (24.73 g; 19.3 mmole) was evaporated and dissolved in dried DMSO (7.5 mL). The two DMSO solutions were mixed and heated to 55° C. overnight. The DMSO solution was placed in dialysis tubing (1000 MWCO, 45 mm flat width×50 cm long) and dialyzed against water for 3 days. The polyalditol macromer solution was filtered and lyophilized to give 7.91 g white solid. A sample of the macromer (49 mg), and DBB (4.84 mg) was dissolved in 0.8 mL DMSO-d₆: ¹H NMR (DMSO-d₆, 400 MHz) δ 7.38 (s, 4H; internal std. integral value of 2.7815), 6.50, 6.19, and 5.93 (doublets, 3H; vinyl protons integral value of 3.0696). The calculated acrylate load of macromer was 0.616 μmoles/mg of polymer.

### EXAMPLE 18

### Maltodextrin-Acrylate Filaments

1,100 milligrams of MD-Acrylate as prepared in Example 11 was placed in an 8 mL amber vial. To the MD-Acrylate was added 1 mg of a photoinitiator 4,5-bis(4-benzoylphenyl-methyleneoxy)benzene-1,3-disulfonic acid (5 mg)(DBDS) and 1 mL of 1× phosphate-buffered saline (PBS). The reagents were then mixed for one hour on a shaker at 37° C. The mixture in an amount of 10 uL was injected, using a 23 gauge needle, into a 22 mm length opaque silicone tube (P/N 10-447-01; Helix Medical, Carpinteria, Calif.). The tubing was placed into a Dymax Lightweld PC-2 illumination system (Dymax Corp.; light intensity 6.5 mW/cm²), 15 cm from light source, illuminated for 270 seconds, and then removed. After illumination, the filament was removed from the silicone tubing by rolling a pencil over the tubing, starting from the back. The filament was firm, which indicated complete polymerization of the MD-Acrylate. No excess liquid was observed. The filament was manipulated with forceps. Maltodextrin filaments were also made from a MD-acrylate solution having concentration of 16.7% solids content (200 mg+1 mL). These are physically firm and same as the composition with MD-acrylate at 52.4% solids content (1,100 mg+1 mL).

### EXAMPLE 19

### Polyalditol-Acrylate Filaments

1,500 milligrams of polyalditol-acrylate as prepared in Example 17 was placed in an 8 ml amber vial. To the polyalditol-acrylate was added 1 mg of DBDS (lyophilized), 15 mg Bovine Serum Albumin, and 200 uL of 1× phosphate-buffered saline (PBS). The reagents were then mixed for one hour on a shaker at 37° C. The mixture in an amount of 10 uL was injected, using a 23 gauge needle, into a 22 mm length opaque silicone tube (P/N 10-447-01; Helix Medical, Carpinteria, Calif.). The tubing was placed into a Dymax Lightweld PC-2 illumination system (Dymax Corp.; light intensity 6.5 mW/cm²), 15 cm from light source, illuminated for 270 seconds, and then removed. After illumination, the filament was removed from the silicone tubing by rolling a pencil over the tubing, starting from the back. The filament was firm, which indicated complete polymerization of the polyalditol-acrylate. No excess liquid was observed. The filament was manipulated with forceps

### EXAMPLE 20

### Amylase Degradation of Maltodextrin-Acrylate Filaments

Maltodextrin-acrylate filaments were synthesized using the 16.7% solids content (200 mg+1 mL) composition and 52.4% solids content (1,100 mg+1 mL) composition as described in Example 18 and were tested for degradation in Amylase solutions. These filaments were placed in microcentrifuge tubes containing 1 mL of either 1×PBS (control), 1×PBS containing alpha-Amylase at 0.121 μg/mL (Sigma; catalog #A6814), or 1×PBS containing alpha-Amylase at 24 μg/mL. The tubes were then placed in an incubator at 37° C.

After 2 days in the PBS with the 0.121 μg/mL alpha-Amylase solution the filament formed from the 16.7% solids content composition filament was completely degraded, and no trace of the filament was observable. The filament formed from the 16.7% solids content composition in PBS (control) showed no signs of degradation.

After 33 days in the 1×PBS containing alpha-Amylase at 0.121 μg/mL, the filament formed from the 52.4% solids content composition had lost some of its initial firmness (as noted by the slightly curled appearance of the filament), but was still completely intact. The filament formed from the 52.4% solids content composition in the PBS with 24 ug Amylase had completely degraded after 48 hours. The filament formed from the 52.4% solids content composition in the PBS showed no signs of degradation.

### EXAMPLE 21

### Maltodextrin-Acrylate Filaments with Bioactive Agent and Release

MD-Acrylate in an amount of 1,100 milligrams of as prepared in Example 11 was placed in an 8 ml amber vial. To the MD-Acrylate was added 1 mg of DBDS (lyophilized), 15 mg Bovine Serum Albumin (representing the bioactive agent; and 1 mL of 1× phosphate-buffered saline (1×PBS). The reagents were then mixed for one hour on a shaker at 37° C. The mixture in an amount of 10 uL was injected, using a 23 gauge needle, into a 22 mm length opaque silicone tube (P/N 10-447-01; Helix Medical, Carpinteria, Calif.). The tubing was placed into a Dymax Lightweld PC-2 illumination system (Dymax Corp.; light intensity 6.5 mW/cm²), 15 cm from light source, illuminated for 270 seconds, and then removed. After illumination, the filament was removed from the silicone tubing by rolling a pencil over the tubing, starting from the back. The filament was firm, which indicated complete polymerization of the MD-Acrylate. No excess liquid was observed.

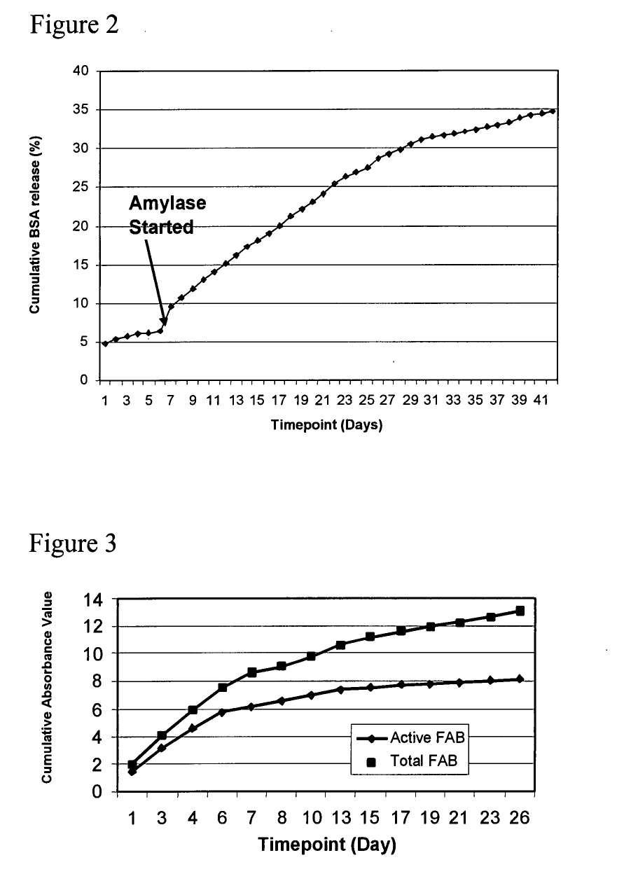

The filament was placed in a 1.7 ml microcentrifuge tube with 1 ml 1×PBS. At daily intervals for 6 days, 150 μL of PBS was removed from each well and placed into a 96 well plate for subsequent analysis. The remaining 850 μL was removed from the sample, and to the tube was added 1 ml of 1×PBS. After 6 days, the filament was placed in a 1.7 ml microcentrifuge tube with 1×PBS containing alpha-Amylase at 0.121 μg/mL. At daily intervals for 35 days, 150 μL of PBS was removed from each well and placed into a 96 well plate for subsequent analysis. The remaining 850 μL was removed from the sample, and to the tube was added 1 ml of fresh 1×PBS containing alpha-Amylase at 0.121 μg/mL. The 96-well plate was analyzed for BSA using the Quanitpro Assay Kit (Sigma). For the first 6 days, there was an initial burst of BSA, followed by a very slow release. After the addition of PBS+Amylase, the rate of BSA release significantly increased, and was relatively constant over the next 35 days. Results are shown in Table 2 and FIG. 2.

TABLE 2

| | Cumulative BSA release |
|---|---|
| Timepoint | (% of Total BSA) |
| 1 | 4.8 |
| 2 | 5.35 |
| 3 | 5.7 |
| 4 | 5.98 |
| 5 | 6.19 |
| 6 | 6.36 |
| 7 | 9.46 |
| 8 | 10.7 |
| 9 | 11.82 |
| 10 | 12.94 |
| 11 | 14.01 |
| 12 | 15.06 |
| 13 | 16.11 |
| 14 | 17.23 |
| 15 | 18.11 |
| 16 | 19.04 |
| 17 | 19.92 |
| 18 | 21.26 |
| 19 | 22.15 |
| 20 | 23.04 |
| 21 | 24.06 |
| 22 | 25.35 |
| 23 | 26.31 |
| 24 | 26.91 |
| 25 | 27.51 |
| 26 | 28.63 |
| 27 | 29.19 |
| 28 | 29.75 |
| 29 | 30.44 |
| 30 | 31.11 |
| 31 | 31.43 |
| 32 | 31.63 |
| 33 | 31.83 |
| 34 | 32.07 |
| 35 | 32.31 |
| 36 | 32.72 |
| 37 | 32.95 |
| 38 | 33.27 |
| 39 | 33.83 |
| 40 | 34.15 |
| 41 | 34.43 |
| 42 | 34.71 |

### EXAMPLE 22

### Polyalditol-Acrylate Filaments with Bioactive Agent and Release

Polyaldtiol-acrylate in an amount of 1,500 mg of as prepared in Example 17 was placed in an 8 ml amber vial. To the PA-Acrylate was added 1 mg of DBDS (lyophilized), 15 mg Bovine Serum Albumin, and 1 mL of 1× phosphate-buffered saline (1×PBS). The reagents were then mixed for one hour on a shaker at 37° C. The mixture in an amount of 10 uL was injected, using a 23 gauge needle, into a 22 mm length opaque silicone tube (P/N 10-447-01; Helix Medical, Carpinteria, Calif.). The tubing was placed into a Dymax Lightweld PC-2 illumination system (Dymax Corp.; light intensity 6.5 mW/cm²), 15 cm from light source, illuminated for 270 seconds, and then removed. After illumination, the filament was removed from the silicone tubing by rolling a pencil over the tubing, starting from the back. The filament was firm, which indicated complete polymerization of the polyalditol-acrylate. No excess liquid was observed. The filament was manipulated with forceps.

The filament was placed in a 1.7 ml microcentrifuge tube with 1 ml PBS containing alpha-Amylase at 0.121 μg/mL. At daily intervals for 15 days, 150 μl of PBS was removed from each well and placed into a 96 well plate for subsequent analysis. The remaining 850 μL was removed from the sample, and to the tube was added 1 ml of fresh PBS containing alpha-Amylase at 0.121 μg/mL. The 96-well plate was analyzed for BSA using the Quanitpro Assay Kit (Sigma).

### EXAMPLE 23

### Maltodextrin-Acrylate Filaments with Bioactive Agent and Release

Maltodextrin filaments were synthesized using a 52.4% solids content (1,100 mg+1 mL) composition as described in Example 21 using an anti-horseradish peroxidase antibody (P7899; Sigma) instead of BSA. The filament contained 800 ug of the anti-horseradish peroxidase antibody. The filament was placed in a 1.7 ml microcentrifuge tube containing 1 ml of 1×PBS containing alpha-Amylase at 0.121 μg/mL. At daily intervals for 5 days, 100 μl of PBS was removed from the sample, placed into a 96 well plate and incubated for 60 minutes at 37° C. The remaining 850 μL was removed from the sample, and replaced with 1 ml fresh 1×PBS containing alpha-Amylase at 0.121 μg/mL. After 1 hour, the plate was washed three times with 1 ml PBS/Tween (Sigma). 150 ul StabilCoat™ Immunoassay Stabilizer (SurModics, Eden Prairie, Minn.) was added to the well and incubated for 30 minutes at room temperature. After 30 minutes, the 96-well plate was washed three times with PBS/Tween. A solution of 0.5 mg/ml Horseradish Peroxidase (Sigma) in 1×PBS (100 uL) was added to the well and incubated for 60 minutes. After 60 minutes, the 96-well plate was washed six times with PBS/Tween. A chromogenic assay was then performed. After 15 minutes, the 96 well plate was analyzed for HRP conjugate on a spectrophotometer (Tecan) at 560 nm absorbance. Detectable Antibody was found at each time point.

### EXAMPLE 24

### Degradation of MD-Acrylate Filament in Vitreal Fluid

A circumferential dissection of the anterior segment (cornea, aqueous humour, lens) of porcine eye was performed, and the vitreous was squeezed out from the globe into a 20 mL amber vial; approx 10 mL total was retrieved from a total of four eyes. Maltodextrin filaments, formed in Example 17, were placed into 2 mL of the vitreous solution, and placed at 37° C. on a rotator plate. The filament formed from the 16.7% solids content (200 mg+1 mL) composition had completely dissolved after 24 hours. The filament formed from the 52.4% solids content (1,100 mg+1 mL) completely degraded after 30 days in the vitreous.

### EXAMPLE 25

### Formation of a Maltodextrin-Acrylate Biodegradable Matrix Using REDOX Chemistry

Two solutions were prepared. Solution #1 was prepared as follows: 250 mg of MD-acrylate as prepared in Example 11 was placed in an 8 mL vial. To the MD-acrylate was added 15 mg ferrous gluconate hydrate (Sigma), 30 mg Ascorbic Acid (Sigma), 67 uL AMPS (Lubrizol) and 1,000 uL deionized water. Solution #2 was prepared as follows: 250 mg of MD-acrylate as prepared in Example 11 was placed in a second 8 mL vial. To this MD-acrylate was added 30 uL AMPS, 80 uL Hydrogen Peroxide (Sigma) and 890 uL 0.1 M Acetate buffer (pH 5.5).

50 uL of Solution #1 was added to a glass slide. 50 uL of solution #2 was added to Solution #1 with slight vortexing. After mixing for 2 seconds, the mixture polymerized and formed a semi-firm gel having elastomeric properties.

### EXAMPLE 26

### Bioactive Agent Incorporation into a MD-Acrylate Matrix

Two solutions were prepared. Solution #1 was prepared as follows: 250 mg of MD-acrylate (as prepared in Example 13) was placed in an 8 ml vial. To the MD-acrylate was added 15 mg Iron (II) Acetate (Sigma), 30 mg Ascorbic Acid (Sigma), 67 ul AMPS (Lubrizol), 75 mg Bovine Serum Albumin (BSA; representing the bioactive agent) and 1,000 μL deionized water. Solution #1 was prepared as follows: 250 mg of MD-acrylate was placed in a second 8 ml vial. To this MD-acrylate was added 30 μL AMPS, 80 μL Hydrogen Peroxide (Sigma), 75 mg BSA and 890 μL Acetate buffer (pH 5.5).

50 μL of Solution #1 was added to a glass slide. 50 μL of solution #2 was added to Solution #1 with slight vortexing. After mixing for 2 seconds, the mixture polymerized and formed a semi-firm gel having elastomeric properties.

### EXAMPLE 27

### Enzyme Degradation of a MD-Acrylate Matrix Formed by Redox

Maltodextrin-acrylate filaments were prepared using the reagents at concentrations as described in Example 25. These filaments were placed in microcentrifuge tubes containing 1 ml either Phosphate Buffered Saline (PBS) or 1×PBS containing alpha-Amylase at 0.121 μg/mL. The tubes were then placed in an incubator at 37° C.

After 4 days in the 1×PBS containing alpha-Amylase at 0.121 μg/mL, the filament formed from the 20% solids composition (250 mg+1 mL) had completely degraded, leaving no trace of the matrix. The matrix in PBS showed no signs of degradation.

### EXAMPLE 28

### FAB Fragment Incorporation and Release from a MD-Acrylate Filament

600 milligrams of MD-Acrylate as prepared in Example 11 was placed in an 8 mL amber vial. To the MD-Acrylate was added 5 mg of DBDS (lyophilized), 10 mg Rabbit Anti-Goat Fragment Antibody (catalog #300-007-003; Jackson Immunological Research, West Grove, Pa.) and 1 mL of 1× phosphate-buffered saline (PBS). The reagents were then mixed for one hour on a shaker at 37° C. The mixture in an amount of 10 μL was pipetted into a 22 mm length opaque silicone tube (P/N 10-447-01; Helix Medical, Carpinteria, Calif.). The tubing was placed into a Dymax Lightweld PC-2 illumination system (Dymax Corp.; light intensity 6.5 mW/cm²), 15 cm from light source, illuminated for 270 seconds, and then removed. After illumination, the filament was removed from the silicone tubing by rolling a pencil over the tubing, starting from the back. The filament was firm and completely crosslinked, with no excess liquid. The filament was placed in a 1.7 mL microcentrifuge tube with 0.5 ml 1×PBS containing alpha-Amylase at 0.121 μg/mL (eluent solution). At predetermined intervals for 17 days, 200 μL of the eluent solution was removed from each tube, and 100 μL was placed into two 96 well plates. The remaining 300 μL were removed from the samples, and replaced with 0.5 mL fresh 1×PBS containing alpha-Amylase at 0.121 μg/mL. The 96 well plates were analyzed for total FAB molecule release and FAB activity using an Enzyme-Linked Immunosorbent Assay (ELISA). Briefly, the 100 μL eluent solution was incubated at 37° C. for one hour and then washed 3× with 2 ml PBS/Tween 20 (Sigma). The wells were blocked with 100 μL StabilCoat™ for 1 hour at room temperature and then washed 3× with 2 mL PBS/Tween 20. 100 uL of either 0.1 ug/mL (in PBS/Tween) HRP-labeled Goat IgG (Jackson Immunological; catalog #005-030-003) for molecule activity or 0.08 ug/mL (in PBS/Tween) HRP-labeled Goat anti-Rabbit IgG (Jackson Immunological; catalog #111-305-003) was incubated for 1 hour at 37° C. The wells were washed 6× with 2 mL PBS/Tween 20. 100 μL of TMB Microwell Peroxidase Substrate System (KPL, Catalog #50-76-00; Gaithersburg, Md.) as added to each well. After 15 minutes, the 96 well plate was analyzed for HRP conjugate on a spectrophotometer (Tecan) at 650 nm absorbance. Detectable Antibody was found at each timepoint. Results are shown in Table 3 and FIG. 3.

TABLE 3
Fab Fragment release ABS values

| | Cumulative Active FAB | Cumulative Total Fab |
|---|---|---|
| Timepoint (Day) | Abs at 650 nm | Abs at 650 nm |
| 1 | 1.37 | 1.97 |
| 3 | 3.12 | 4.07 |
| 4 | 4.54 | 5.87 |
| 6 | 5.69 | 7.54 |
| 7 | 6.12 | 8.60 |
| 8 | 6.53 | 9.01 |
| 10 | 6.94 | 9.79 |
| 13 | 7.34 | 10.64 |
| 15 | 7.54 | 11.18 |
| 17 | 7.71 | 11.62 |
| 19 | 7.81 | 11.92 |
| 21 | 7.90 | 12.28 |
| 23 | 8.00 | 12.68 |
| 26 | 8.09 | 13.11 |

### EXAMPLE 29

### Rabbit Antibody Incorporation and Release from a MD-Acrylate Filament

600 Milligrams of MD-Acrylate as prepared in Example 11 was placed in an 8 ml amber vial. To the MD-Acrylate was added 5 mg of DBDS (lyophilized), 16 mg Rabbit Antibody Anti-HRP (Sigma; catalog #P7899) and 1 ml of 1× phosphate-buffered saline (PBS). The reagents were then mixed for one hour on a shaker at 37° C. The mixture in an amount of 10 μL was pipetted into a 22 mm length opaque silicone tube (P/N 10-447-01; Helix Medical, Carpinteria, Calif.). The tubing was placed into a Dymax Lightweld PC-2 illumination system (Dymax Corp.; light intensity 6.5 mW/cm²), 15 cm from light source, illuminated for 270 seconds, and then removed. After illumination, the filament was removed from the silicone tubing by rolling a pencil over the tubing, starting from the back. The filament was firm and completely crosslinked, with no excess liquid.

The filament was placed in a 1.7 ml microcentrifuge tube with 0.5 ml 1×PBS containing alpha-Amylase at 0.121 μg/mL (eluent solution). At predetermined intervals for 25 days, 200 μl of the eluent solution was removed from each tube, and 100 μL was placed into two 96 well plates. The remaining 300 μl were removed from the samples, and replaced with 0.5 ml fresh 1×PBS containing alpha-Amylase at 0.121 μg/mL. The 96 wellplates were analyzed for total Rabbit Antibody molecule release and activity using an Enzyme-Linked Immunosorbent Assay (ELISA). Briefly, the 100 μL eluent solution was added to the wells and incubated at 37 degrees C. for one hour and then washed 3× with 2 ml PBS/Tween 20 (Sigma). The wells were blocked with 100 μL StabilCoat™ (SurModics) for 1 hour at room temperature and then washed 3× with 2 ml PBS/Tween 20. 100 μL of either 0.1 ug/ml (in PBS/Tween) HRP (Sigma; catalog #P8375) for molecule activity or 0.08 ug/ml (in PBS/Tween) HRP-labeled Goat anti-Rabbit IgG (Jackson Immunological; catalog #111-305-003) was incubated for 1 hour at 37 degrees C. The wells were washed 6× with 2 ml PBS/Tween 20. 100 μL of TMB Microwell Peroxidase Substrate System (KPL, Catalog #50-76-00; Gaithersburg, Md.) was added to each well. After 15 minutes, the 96 well plate was analyzed for HRP conjugate on a spectrophotometer (Tecan) at 650 nm absorbance. Detectable Antibody was found at each time point.

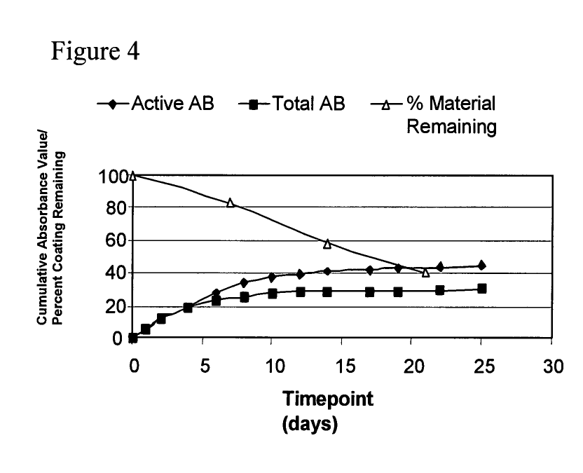

Results are Shown in Table 4 and FIG. 4.

TABLE 4

| | Cumulative | Cumulative | | Maximum |
|---|---|---|---|---|
| | Active IgG | Total IgG | MD-acrylate | theoretical |
| Timepoint | release (%) | release (%) | coating | total IgG |
| (Day) | (ELISA) | (ELISA) | remaining (%) | release (%) |
| 1 | 5.56 | 5.31 | | |
| 2 | 12.13 | 11.94 | | |
| 4 | 18.38 | 19.13 | | |
| 6 | 27.75 | 22.88 | | |
| 7 | | | 83 | 17 |
| 8 | 33.50 | 25.44 | | |
| 10 | 37.63 | 27.44 | | |
| 12 | 39.50 | 28.31 | | |
| 14 | 40.75 | 28.57 | 59 | 31 |
| 17 | 41.75 | 28.76 | | |
| 19 | 42.75 | 28.98 | | |
| 21 | | | 40 | 60 |
| 22 | 43.44 | 29.67 | | |
| 25 | 44.31 | 30.67 | | |

### EXAMPLE 30

### Preparation of Acylated Maltodextrin (Butyrylated-MD)

Maltodextrin having pendent butyryl groups were prepared by coupling butyric anhydride at varying molar ratios.

To provide butyrylated-MD (1 butyl/4 glucose units, 1:4 B/GU) the following procedure was performed. Maltodextrin (MD; Aldrich; 11.0 g; 3.67 mmole; DE (Dextrose Equivalent): 4.0-7.0) was dissolved in dimethylsulfoxide (DMSO) 600 mL with stirring. The size of the maltodextrin was calculated to be in the range of 2,000 Da-4,000. Once the reaction solution was complete, 1-methylimidazole (Aldrich; 2.0 g, 1.9 mls) and butyric anhydride (Aldrich; 5.0 g, 5.2 mls) was added with stirring. The reaction mixture was stirred for four hours at room temperature. After this time, the reaction mixture was quenched with water and dialyzed against DI water using 1,000 MWCO dialysis tubing. The butyrylated starch was isolated via lyophilization to give 9.315 g (85% yield). NMR confirmed a butyrylation of 1:3 B/GU (1.99 mmoles butyl/g sample).

To provide butyrylated-MD (1:8 B/GU), 2.5 g (2.6 mL) butyric anhydride was substituted for the amount of butyric anhydride described above. A yield of 79% (8.741 g) was obtained. NMR confirmed a butyrylation of 1:5 B/GU (1.31 mmoles butyl/g sample).

To provide butyrylated-MD (1:2 B/GU), 10.0 g (10.4 mL) butyric anhydride was substituted for the amount of butyric anhydride described above. A yield of 96% (10.536 g) was obtained. NMR confirmed a butyrylation of 1:2 B/GU (3.42 mmoles butyl/g sample).

### EXAMPLE 31

### Preparation of Acrylated Acylated Maltodextrin (Butyrylated-MD-Acrylate)

Preparation of an Acylated Maltodextrin Macromer Having Pendent Butyryl and acrylate groups prepared by coupling butyric anhydride at varying molar ratios.

To provide butyrylated-MD-acrylate (1 butyl/4 glucose units, 1:4 B/GU) the following procedure was performed. MD-Acrylate (Example 11; 1.1 g; 0.367 mmoles) was dissolved in dimethylsulfoxide (DMSO) 60 mL with stirring. Once the reaction solution was complete, 1-methylimidazole (0.20 g, 0.19 mls) and butyric anhydride (0.50 g, 0.52 mls) was added with stirring. The reaction mixture was stirred for four hours at room temperature. After this time, the reaction mixture was quenched with water and dialyzed against DI water using 1,000 MWCO dialysis tubing. The butyrylated starch acrylate was isolated via lyophylization to give 821 mg (75% yield, material lost during isolation). NMR confirmed a butyrylation of 1:3 B/GU (2.38 mmoles butyl/g sample).

### EXAMPLE 32

### Preparation of Acrylated Acylated Maltodextrin (Butyrylated-MD-Acrylate)

Maltodextrin having pendent butyryl and acrylate groups prepared by coupling butyric anhydride at varying molar ratios.

To provide butyrylated-MD-acrylate the following procedure is performed. Butyrylated-MD (Example 31; 1.0 g; 0.333 mmole) is dissolved in dimethylsulfoxide (DMSO) 60 mL with stirring. Once the reaction solution is complete, a solution of EA-NCO from Example 10 (353 mg; 2.50 mmole) is evaporated and dissolved in dried DMSO 1.0 mL). The two DMSO solutions are mixed and heated to 55° C. overnight. The DMSO solution is placed in dialysis tubing (1000 MWCO) and dialyzed against water for 3 days. The macromer solution is filtered and lyophilized to give a white solid.

### EXAMPLE 33

### Preparation of Biodegradable Ocular Implants, Fab Fragment Incorporation, Release, and Detection from a MD-Acrylate Filament

1,500 milligrams (Formulation 1) of MD-acrylate as prepared in Example 11 was placed in an 8 mL amber vial. To the MD-acrylate was added 5 mg of a photoinitiator 4,5-bis(4-benzoylphenyl-methyleneoxy)benzene-1,3-disulfonic acid (DBDS), 65 mg of Rabbit anti Goat IgG, F(ab) (F(ab); Lampire Biological Laboratories; Pipersville, Pa.) and 1 mL of modified PBS (0.01M Phosphate, 0.015M NaCl). The reagents were then mixed for 4 hours on a shaker at room temperature. The mixture in an amount of 20 μL was injected, using a 1 ml syringe, into a 18 mm length of UV-transmissive silicone tubing (0.64 mm ID; P/N 60-011-03; Helix Medical, Carpinteria, Calif.). The tubing was capped on both ends using binder clips and placed into a Dymax Lightweld PC-2 illumination system (Dymax Corp.; light intensity 1.5 mW/cm2), 15 cm from light source, illuminated for 75 seconds, flipped 180 degree, illuminated for an additional 75 seconds, and then removed. After illumination, the tubing was cut in lengths of 0.65 cm. The filaments were pushed from the tubing using a 0.018″ stainless steel rod into a 1.5 ml eppendorf (VWR). The filaments were firm, which indicated complete polymerization of the MD-Acrylate. No excess liquid was observed. The filaments were manipulated with forceps. The filaments were completely dried at 4° C. overnight, weighed on a microbalance (UMX2, Mettler Toledo, Columbus, Ohio), and stored at 4° C. until use.

Maltodextrin filaments containing F(ab) were also made from a MD-acrylate solution having concentration of solids content of 52.4% (1,100 mg+1 mL) (formulation 2). These are physically firm and the same as those made from solution having a solids content of 60% (1,500 mg+1 mL).

Maltodextrin filaments without F(ab) were also made from MD-acrylate solution having concentration of solids content of 52.4% (1,100 mg+1 mL; Control 1) or 60% (1,500 mg+1 mL; Control 2).

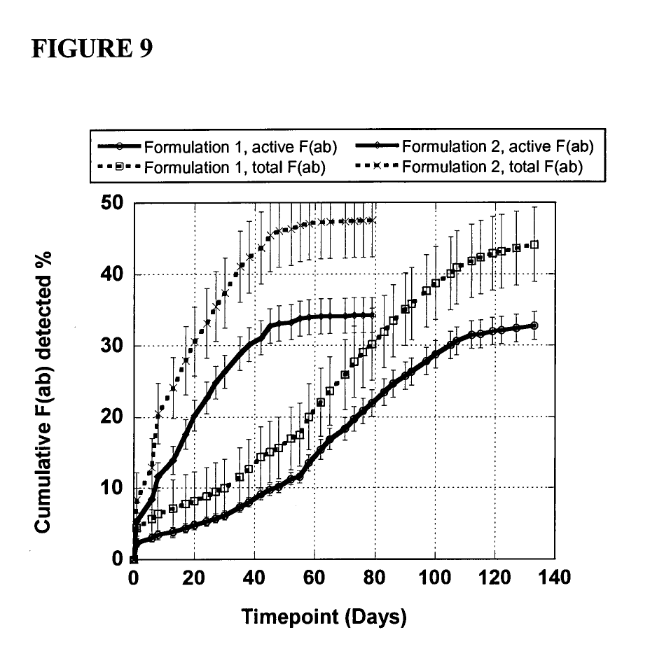

To evaluate in vitro F(ab) elution, filaments were placed in 0.6 mL microcentrifuge tubes (VWR) with 0.5 ml 1×PBS containing alpha-Amylase (Catalog #A6380; Sigma) at 0.121 μg/mL and bovine serum albumin (BSA; Catalog #A7906Sigma,) (eluent solution). At predetermined intervals for 133 days (formulation 1) or 79 days (formulation 2), 200 μL of the eluent solution was removed from each tube, diluted with a known volume of 1×PBS, and analyzed for either total F(ab) molecule release or F(ab) molecule activity using an Enzyme-Linked Immunosorbent Assay (ELISA). The results of the elution over the time interval is represented in FIG. 9.

Briefly, the wells of 96-well plates were first coated with either a goat IgG (Sigma, St. Louis, Mo.; catalog# 15256) solution for F(ab) activity or donkey anti-rabbit IgG (Rockland Immunochemicals, Gilbertsville, Pa.; catalog# 611-703-127) solution for total F(ab) detection. The solutions incubated for 90 minutes at room temperature, and then washed 3× with 300 μL PBS/Tween 20 (Sigma). The wells were blocked with 200 μL StabilCoat (SurModics, Eden Prairie, Minn.) for 1 hour at room temperature and then washed 3× with 300 μL PBS/Tween 20. A 100 μl aliquot of elution solution (from the elution of F(ab) from the MD filament) was added to the appropriate wells and incubated for 1 hour at room temperature, and then washed 3× with PBS/Tween 20. A 100 μL sample of donkey anti-rabbit IgG HRP (Rockland Immunochemicals, Gilbertsville, Pa.; catalog# 611-703-127) was added to each well and incubated for 1 hour at room temperature. The wells were washed 4× with 300 μL PBS/Tween 20. A 100 μL of TMB Microwell Peroxidase Substrate System (KPL, catalog# 50-76-00; Gaithersburg, Md.) was added to each well. For kinetic assays, the TMB substrate produces a blue color upon reaction with peroxidase. After 15 minutes, the 96-well plate was analyzed for HRP conjugate on a spectrophotometer (Molecular Devices) at 650 nm absorbance. For endpoint analysis, addition of an acidic stop solution will halt color development and turn the TMB substrate yellow. Alternatively, after 15 minutes, 100 μL of a 1N HCl solution was added to the well to stop the reaction. Absorption was then measured at 450 nm.

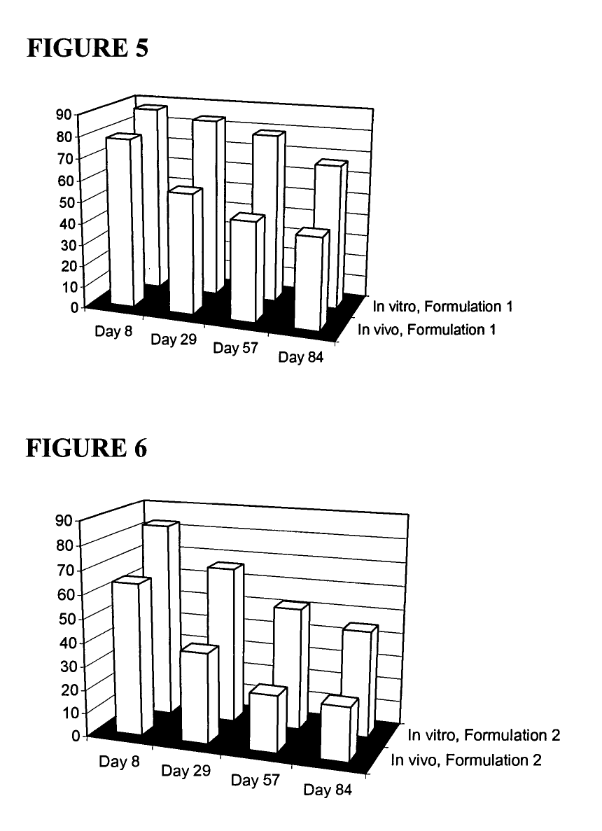

For in vitro filament mass loss evaluation, filaments were placed in 0.6 mL microcentrifuge tubes (VWR) with 0.5 ml 1×PBS containing alpha-Amylase (Catalog #A6380; Sigma) at 0.121 μg/mL and bovine serum albumin (BSA; Sigma, catalog #A7906) (eluent solution). At predetermined timepoints through 84 days for both formulations, all of the eluent solution was removed from each tube, the filaments washed with 500 μL deionized water, and the water removed. The filaments were completely dried and then weighed on a microbalance (UMX2, Mettler Toledo, Columbus, Ohio). Percent mass remaining was calculated by dividing the filament weight at each timepoint by the initial weight (not exposed to alpha-amylase) of the same filament (n=5/timepoint). The results of the mass loss over the time interval is represented in FIG. 5 (formulation 1) and 6 (formulation 2).

### EXAMPLE 34

### Implantation of Biodegradable Ocular Implants

Dutch-belted rabbits were used as animal models for implantation of the biodegradable ocular implants. The study provided information on the pharmacokinetics and safety of different maltodextrin-based ocular implants up to 12 weeks following intravitreal implantation.

Test implants were formulated with either 1500 mg/mL maltodextrin (Test Article 1 (formulation 1), as prepared in Example 33) and Rabbit anti goat IgG Fab (F(ab)), or 1000 mg/mL maltodextrin (Test Article 2 (formulation 2), as prepared in Example 33) and F(ab). Rods not containing F(ab) fragments were used as the corresponding control articles (Control 1 and Control 2, as prepared in Example 33).

Test Article 1 and Test Article 2 were intravitreally implanted in the left and right eyes, respectively, of 26 female rabbits. Control 1 and Control 2 were intravitreally implanted in the right and left eyes, respectively, of four female rabbits. Ophthalmic examinations (slit lamp and indirect opthalmoscopy) and intraocular pressure measurements (IOP) were conducted on Days 3, 8, 29, 56/57/58, and 84/85. All four rabbits implanted with control articles and two of the rabbits implanted with test articles were euthanized on Day 29 or Day 84/85; their globes were histopathologically evaluated. The other 24 rabbits implanted with test articles were euthanized on Day 8, 29, 57, or 84; the implanted articles, vitreous humor, and sclera/retina/choroid complexes were collected from their eyes and used for pharmacokinetics analyses.

There was no mortality of the rabbits in the study. Following implantation of the biodegradable rods (in studies involving rabbits with and without the presence of antibody in the rod) the rabbit eyes were assessed for the following physiological responses: conjunctival discharge, conjunctival congestion, conjunctival swelling, aqueous flare, pupil response, vitreal opacity, vitreal hemorrhage, retinal detachment, and retinal scarring. Pathological analysis from both the Day 29 and Day 84/85 durations revealed that these physiological responses were quite limited.

Treatment efficacy for bioactive agents delivered from the implants can also be measured in a VEGF-induced model of retinal vascular leakage in rabbits.

Details of the animal study are as follows.

Animals

Thirty female Dutch Belted rabbits were obtained from Covance (Denver, Pa.). Animals were 12-13 months old and weighed 1.88-2.80 kg at the time of dosing. Animal husbandry was carried out using approved protocols. Prior to placement on study, a physical examination was performed on each animal. Each animal underwent a pre-treatment ophthalmic examination (slit lamp and indirect opthalmoscopy), performed by a board-certified veterinary ophthalmologist. Prior to dosing, 30 animals were weighed and randomly assigned to eight treatment groups. Animals were fasted at least two hours prior to implantation.

Pharmaceutical Administration

Neomycin/Polymyxin/Bacitracin (NPB) Ophthalmic Ointment was placed in both eyes of each animal once daily on the day of intravitreal implantation (Day 1) and two days after intravitreal implantation (Days 2 and 3). Animals were anesthetized with an injection of ketamine (100 mg/mL) at 35 mg/kg plus xylazine (100 mg/mL) at 7 mg/kg either via intramuscular or intravenous injection. Both eyes of each animal were prepared for implantation as follows: Approximately 20 minutes prior to surgery, two drops of 1% tropicamide were placed into each eye. Ten minutes prior to surgery, two drops of phenylephrine hydrochloride 2.5% were placed into each eye. Eyes were moistened with an ophthalmic Betadine solution. After five minutes, the Betadine was washed out of the eyes with sterile saline. Finally, proparacaine hydrochloride 0.5% (1-2 drops) was delivered to each eye. Eyes were positioned under the operating microscope with a wire lid speculum and draped using Steridrape. For analgesia, animals were administered Buprenorphine at 0.02 mg/kg subcutaneously prior to implantation.

Implantation

For the intravitreal implantation procedure, a small peritomy was made at the superior temporal quadrant of one eye. A sclerotomy was created with a 20-gauge MVR blade, 1-2 mm posterior to the limbus in the superior temporal quadrant. The test or control article was inserted through the sclerotomy, close to the vitreal base, using surgical microforceps. Once the article was fully implanted, the sclerotomy and conjunctival opening were closed with Vicryl 7-0 absorbable sutures. The animal was repositioned and the opposite eye was similarly implanted with the appropriate article. NPB Ophthalmic Ointment was applied to the eye following the implantation procedure.

In an alternative implantation method, the ocular implant is placed within the hollow bore of a 20-25 gauge needle for delivery to the eye. The piercing action of the needle creates a transconjunctival sclerotomy. A plunger is placed into the needle bore proximal to the implant and expels the implant from the needle bore into the vitreous. The needle is then withdrawn from the eye. Using the 25 gauge needle (or smaller), the wound is self-sealing and requires no sutures.

Ophthalmic Observations

Ophthalmic observations (slit lamp and indirect opthalmoscopy) were performed on both eyes of each remaining animal on Days 3, 8, 29, 56/57, and 84/85. Eyes were dilated with a mydriatic agent (1% tropicamide solution) to sufficiently view the retina and vitreous. Intraocular pressure (IOP) was determined for both eyes of each remaining animal on Days 3, 8, 29, 57/58, and 84/85. IOP was evaluated with a Medtronic Solan Model 30 classic pneumatonometer.

Tissue Preparation and Analysis

Animals were euthanized with an intravenous injection of commercial euthanasia solution according to a standard protocol. Eyes designated for safety analysis were prepared as follows: Both globes were enucleated and placed into Davidson's solution for approximately 24 hours. Following the 24-hour period, eyes were transferred to 70% ethanol. The time that eyes were placed into Davidson's solution and the time of removal were recorded. Globes were then submitted for histopathological evaluation. Eyes designated for pharmacokinetics analysis were prepared as follows: Both globes were enucleated and frozen at approximately −70° C. in liquid nitrogen. The following tissues were collected from all eyes and their weights recorded: The vitreous humor was collected with care not to contaminate with ciliary body or retina cells. The sclera, retina, and choroid were collected as a single complex. For each eye, the time that necropsy/tissue collection was completed was recorded. All tissue samples were stored at approximately −70° C. During the collection of vitreous humor from each eye, the test article was explanted from the eye and placed in a dry, labeled eppendorf tube. Test articles explanted were either stored at 4° C. or in the dark at −70° C. prior to analysis.

Explant Analysis

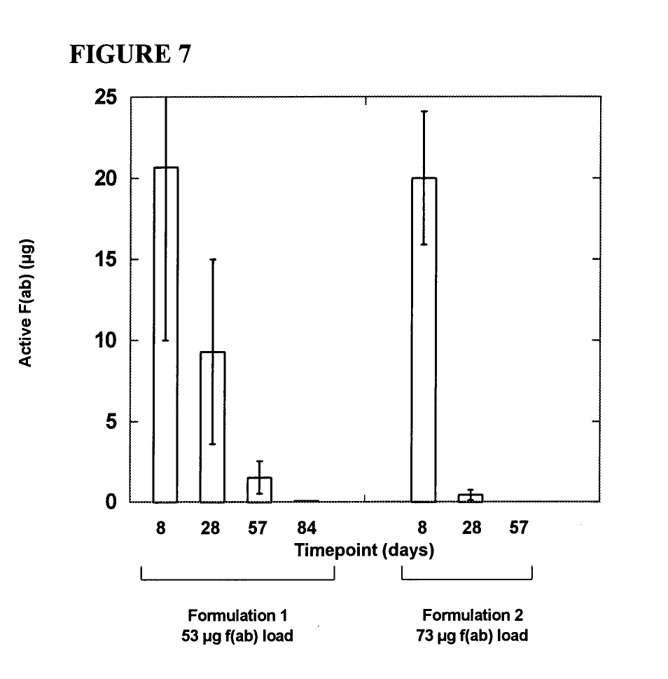

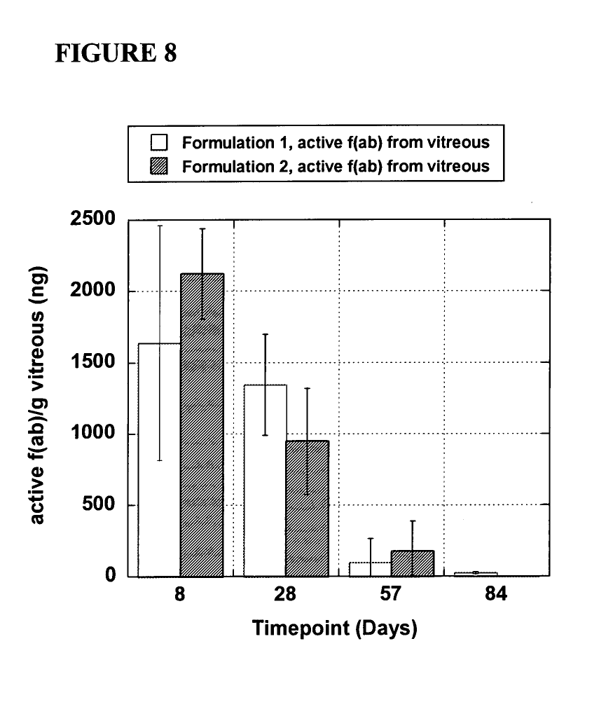

At 7, 28, 56 and 84 day timepoints, the devices ((Formulation 1, with 53 μg of F(ab); Formulation 2 with 73 μg F(ab); n=6/formulation/timepoint) were explanted and assayed for remaining active and total F(ab) using ELISA (as described in Example 33), the data which is represented in FIG. 7. Vitreous samples at these time points were similarly assayed via ELISA for active F(ab), the data which is represented in FIG. 8.

For explanted filament mass loss evaluation, explanted filaments were completely dried, excess adherent tissue was removed via a razor blade, and then weighed on a microbalance (UMX2, Mettler Toledo, Columbus, Ohio). Percent mass remaining was calculated by dividing the filament weight at each timepoint by the initial weight of the filament, with the data represented in FIG. 5 (formulation 1) and 6 (formulation 2).

### EXAMPLE 35

### Preparation of Molecular Weight Fractionated Maltodextrin (Fractionated MD)

Maltodextrin having molecular weight ranges were prepared by diafiltration of the maltodextrin using ultrafiltration membranes with differing pore sizes.

To provide fractionated MD the following procedure was performed. Maltodextrin (MD; Grain Processing Corp, Muscatine, Iowa; 1 kg; DE (Dextrose Equivalent): 9-12) was dissolved in 9000 mL deionized water with stirring. The maltodextrin can be diafiltered using one cassette holder or via a dual diafiltration system ran simultaneously. The ten liters of MD solution was kept at a constant volume of ten liters (retentate) and diafiltered versus a 30 K cassette. A total of 100 liters of permeate was collected. The 100 liters of permeate that was collected was concentrated versus 1 K cassettes down to a retentate volume of 10 liters. The fractionated MD (1-30 K) was isolated via lyophilization to give 546.3 g (55% yield). GPC-MALLS confirmed a MW_{AVE} 7900, starting material MW_{AVE} 11,300.

To provide fractionated MD (<30 K): 30 g was fractionated as above; 3 liters permeate was collected and lyophilized. A yield of 79% (23.6 g) was obtained. GPC-MALLS confirmed a MW_{AVE} 6,876, starting material MW_{AVE} 15,400.

To provide fractionated MD (1-10 K): 100 g was fractionated as above using a 10 K membrane; 10 liters permeate was concentrated vs. 1 K cassette to 1 liter retentate volume. A yield of 64% (63.9 g) was obtained. GPC-MALLS confirmed a MW_{AVE} 7,300, starting material MW_{AVE} 15,400

To provide fractionated MD (5-30 K): 100 g was fractionated as above; 10 liters permeate was concentrated vs. 5 K cassette to 1 liter retentate volume. A yield of 49% (48.6 g) was obtained. GPC-MALLS confirmed a MW_{AVE} 17,860, starting material MW_{AVE} 11,300.

To provide fractionated MD (10-30 K): 300 g was fractionated as above; 30 liters permeate was concentrated vs. 10 K cassette to 3 liter retentate volume. A yield of 20% (60.5 g) was obtained. GPC-MALLS confirmed a MW_{AVE} 25,000, starting material MW_{AVE} 11,300.

### EXAMPLE 36

### Preparation of Maltodextrin-Methacrylate Macromer (MD-Methacrylate)

Maltodextrin-methacrylate was prepared as follows: 1-30 K maltodextrin or 5-30 K maltodextrin (as prepared in example 35) was dissolved in dimethylsulfoxide (DMSO) 1,000 mL with stirring. Once the reaction solution was complete, 1-methylimidazole (Aldrich; 2.0 g, 1.9 mL) followed by methacrylic-anhydride (Aldrich; 38.5 g) were added with stirring. The reaction mixture was stirred for one hour at room temperature. After this time, the reaction mixture was quenched with water and dialyzed against DI water using 1,000 MWCO dialysis tubing. The MD-methacrylate was isolated via lyophilization to give 63.283 g (63% yield). The calculated methacrylate load of macromer was 0.56 μmoles/mg of polymer for the 1-30 K MD-methacrylate, and 0.54 μmoles/mg of polymer for the 5-30 K MD-methacrylate.

### EXAMPLE 37

### Preparation of Biodegradable Ocular Implants, FAB Fragment Incorporation, Release, and Detection from a MD-Methacrylate Filament

1,300 milligrams of 1-30 K (formulation 1) or 5-30 K (formulation 2) MD-methacrylate, as prepared in Example 36, was placed in an 8 mL amber vial. To the MD-methacrylate was added 5 mg of a photoinitiator 4,5-bis(4-benzoylphenyl-methyleneoxy) benzene-1,3-disulfonic acid (DBDS), 90 mg of Rabbit anti Goat IgG, F(ab) (F(ab); Lampire Biological Laboratories; Pipersville, Pa.) and 1 mL of modified PBS (0.01M Phosphate, 0.015M NaCl). The reagents were then mixed for 4 hours on a shaker at room temperature. The mixture in an amount of 20 μL was injected, using a 1 ml syringe, into a 18 mm length opaque silicone tube (0.64 mm ID; P/N 60-011-03; Helix Medical, Carpinteria, Calif.). The tubing was capped on both ends using binder clips and placed into a Dymax Lightweld PC-2 illumination system (Dymax Corp.; light intensity 1.5 mW/cm2), 15 cm from light source, illuminated for 60 seconds, flipped 180 degree, illuminated for an additional 60 seconds, and then removed. After illumination, the tubing was cut in lengths of 0.65 cm. The filaments were pushed from the tubing using a 0.018″ stainless steel rod into a 1.5 ml eppendorf (VWR). The filaments were firm, which indicated complete polymerization of the MD-methacrylate. No excess liquid was observed. The filaments were manipulated with forceps. The filaments were allowed to dry at 4° C. overnight, weighed on a microbalance (UMX2, Mettler Toledo, Columbus, Ohio), and stored at 4° C. until use.

### EXAMPLE 38

### Implantation of Biodegradable Ocular Implants

In another animal study similar to that described in Example 34, Dutch-belted rabbits were used as animal models for implantation of the biodegradable ocular implants prepared according to Example 37.

Explant Analysis

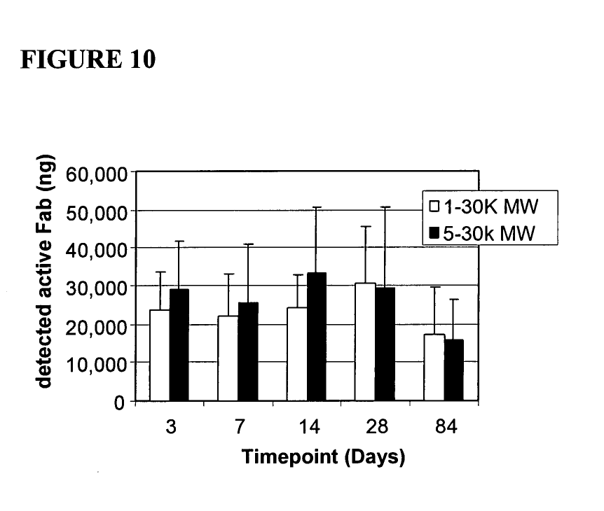

At 3, 7, 14, 28, and 84 day timepoints, the devices (Formulation's 1 and 2, with ˜75 μg F(ab)/device; n=5/formulation/timepoint) were explanted and assayed for remaining active and total F(ab) using ELISA (as described in example 33), the data which is represented in FIG. 10.

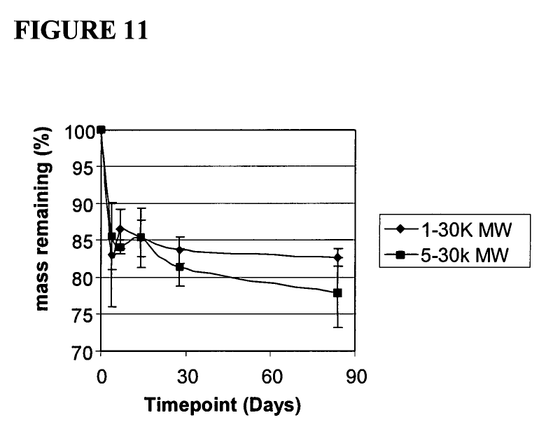

For explanted filament mass loss evaluation, explanted filaments were completely dried, gross excess adherent tissue was removed via a razor blade, and then weighed on a microbalance (UMX2, Mettler Toledo, Columbus, Ohio). Percent mass remaining was calculated by dividing the filament weight at each timepoint by the initial weight of the filament, with the data represented in FIG. 11.

We claim:

1. A biodegradable implant for delivery of a bioactive agent to the interior of the eye, wherein the implant comprises a matrix comprising a biodegradable polymer and a bioactive agent which is capable of releasing a therapeutically effective amount of bioactive agent in the interior of the eye after a period of 30 days from implantation.

2. The biodegradable implant of claim 1 which is configured so that it can be placed at a location in the inner eye using a sutureless procedure.

3. The biodegradable implant of claim 1 wherein the biodegradable polymer comprises a natural biodegradable polysaccharide.

4. The biodegradable implant of claim 1 wherein the matrix is formed from biodegradable polymers crosslinked via pendent polymerized groups.

5. The biodegradable implant of claim 3 wherein the biodegradable polymer comprises a linear polymer having repeating glucopyranose units that are joined by α-1,4 linkages.

6. The biodegradable implant of claim 1 wherein the biodegradable polymer has an average molecular weight of 50,000 Da or less.

7. The biodegradable implant of claim 6 wherein the biodegradable polymer has an average molecular weight in the range of 1000 Da to about 10,000 Da.

8. The biodegradable implant of claim 1 wherein the bioactive agent is a hydrophilic compound.

9. The biodegradable implant of claim 1 wherein the bioactive agent is a polypeptide.

10. The biodegradable implant of claim 9 wherein the bioactive agent is an antibody or an antibody fragment.

11. The biodegradable implant of claim 1 wherein the bioactive agent is selected from the group consisting of alemtuzumab, gemtuzumab, rituximab, ibritumomab, tositumomab, edrecolomab, cetuximab, and bevacizumab, ranibizumab, satumomab, pertuzumab, and daclizumab.

12. The biodegradable implant of claim 1 wherein the bioactive agent is selected from the group consisting of antiproliferative agents, anti-inflammatory agents, anti-angiogenic agents, hormonal agents, antibiotics, and neurotrophic factors.

13. The biodegradable implant of claim 1 wherein the biodegradable polymer is present in the implant in an amount of 87.5% wt or greater.

14. The biodegradable implant of claim 13 wherein the biodegradable polymer is present in the implant in an amount of 90% wt or greater.

15. The biodegradable implant of claim 1 wherein the bioactive agent is present in the implant in an amount of up to 12.5% wt.

16. The biodegradable implant of claim 15 wherein the bioactive agent is present in the implant in the range of 5% wt to 11% wt.

17. A method for administering a bioactive agent to the inner eye, the method comprising the steps of:
providing a biodegradable implant comprising a matrix comprising a biodegradable polymer and a bioactive agent, wherein the implant is configured so that it can be placed at a location in the inner eye using a sutureless surgical procedure,
implanting the implant in the inner eye, and
maintaining the implant in the inner eye, wherein the implant releases a therapeutically useful amount of bioactive agent in the inner eye after a period of 30 days from the step of implanting.

18. The method of claim 17, wherein the step of implanting comprises implanting the biodegradable implant in the inner eye in a sutureless process.

19. The method of claim 17, wherein the step of maintaining, the implant releases a therapeutically useful amount of bioactive agent in the inner eye after a period of 60 days from the step of implanting.

20. The method of claim 19, wherein the step of maintaining, the implant releases a therapeutically useful amount of bioactive agent in the inner eye after a period of 120 days from the step of implanting.

21. The method of claim 17 performed to treat an ocular condition or indication selected from the group consisting of retinal detachment, vascular occlusion, retinitis pigmentosa, proliferative vitreoretinopathy, diabetic retinopathy, uveitis, choroiditis, retinitis, age-related macular degeneration, vascular diseases, tumor growth, and neoplasms.

22. A method for administering a bioactive agent to the inner eye, the method comprising the steps of:
providing a biodegradable implant comprising a matrix of natural biodegradable polysaccharides and a bioactive agent, and
implanting the implant in the interior of the eye, wherein the implant releases a therapeutically useful amount of bioactive agent in the inner eye.

23. A bioactive agent-releasing biodegradable ocular implant comprising a matrix of natural biodegradable polysaccharides and bioactive agent within the matrix.

24. A kit for placing a biodegradable implant in the inner eye, the kit comprising:
a biodegradable implant comprises a matrix comprising a biodegradable polymer and a bioactive agent which is capable of releasing a therapeutically effective amount of bioactive agent in the inner eye after a period of 30 days from implantation, and
an insertion instrument to provide the implant to a target site within the inner eye.

25. The kit of claim 24, wherein the insertion instrument comprises a needle having a size of 25 gauge or smaller in which the implant can be placed.
